(19) United States
(12) Patent Application Publication
(10) Pub. No.: US 2008/0019910 A1
(43) Pub. Date: Jan. 24, 2008

(54) Cancer Treatment and Cancer Treatment Efficacy Prediction by Blocking and Detecting Protease Inhibitors

(76) Inventors: Maria Unni Romer, Copenhagen S (DK); Ulrik Axel Lademann, Soborg (DK); Kenneth Francis Hofland, Birkerod (DK); Peter Buhl Jensen, Farum (DK); Marion Ellen Meijer Van Gelder, Ouddorp (NL); Johannes Albert Foekens, Capelle aan den ljssel (NL); Anne-Sofie Schrohl Rasmussen, Copenhagen S (DK); Nils Age Brunner, Hellerup (DK); Pernille Autzen Usher, Copenhagen O (DK)

Correspondence Address: ROBERTS, MLOTKOWSKI & HOBBES, P. O. BOX 10064, MCLEAN, VA, 22102-8064, US

(21) Appl. No.: 10/594,999

(22) PCT Filed: Mar. 30, 2005
(86) PCT No.: PCT/DK05/00218

§ 371 Date: Sep. 12, 2007

Related U.S. Application Data
(60) Provisional application No. 60/558,123, filed on Apr. 1, 2004.

(30) Foreign Application Priority Data
Mar. 30, 2004 (DK) PA 2004 00506

(51) Int. Cl. A61K 49/00; A61K 31/337; A61K 31/7052; A61K 33/34; C12Q 1/68; C12Q 1/02; A61K 38/02; A61K 39/395

(52) U.S. Cl. 424/9.2; 424/130.1; 424/141.1; 424/630; 435/29; 435/40.52; 435/6; 514/2; 514/44; 514/449; 514/789

(57) ABSTRACT

Disclosed is a method for improving cancer therapy that relies on induction of apoptosis in malignant cells. It has been found that docking of protease inhibitors PAI-1 and TIMP-1 renders malignant cells expressing these inhibitors more sensitive to apoptosis, whereas non-malignant cells do not change their sensitivity to apoptosis induction. It is therefore possible to increase the effect of various anti-cancer treatments in a rational manner and to predict whether or not an apoptosis-inducing cancer treatment will be effective in a patient or not. The invention also provides for methods of identifying agents that inhibit the apoptosis sensitivity modulating effects of protease inhibitors and to methods of identifying anti-cancer compounds that are not dependent on an apoptosis inducing mechanism which can be modulated by protease inhibitors.

### FIELD OF THE INVENTION

The present invention relates to the field of cancer therapy. In particular, the present invention relates to methods to increase the sensitivity of malignant cells but not non-malignant cells to various types of anti-cancer agents and anti-cancer treatments. In particular, the present invention relates to improvements in therapy of cancer patients and in improvements in prediction of cancer therapy efficacy.

### BACKGROUND OF THE INVENTION

Apoptosis

Apoptosis, or programmed cell death, is a cell suicide mechanism that enables multicellular organisms to maintain tissue homeostasis and to eliminate cells that threaten the survival of an animal. Deregulation of apoptosis is observed in various diseases such as neurodegenerative diseases where excess cell death is pronounced and cancers where apoptosis is inhibited.

Apoptosis can be triggered by a variety of stimuli, including activation of cell surface death receptors (Fas, TRAIL-R1/R2, TNF-R1 etc.), anticancer agents, irradiation, lack of survival factors, and ischemia. Even though the initial signalling pathways induced by various stimuli can be very different, the signalling cascades induced by most of them finally converge into a common apoptotic pathway characterized by the activation of a family of cysteine proteases, known as the caspases. Apoptosis can be induced by two major caspase activation pathways, the “extrinsic cell death pathway” and the “intrinsic cell death pathway”. Stimulation of these two pathways induces the activation of initiator caspases, which subsequently activate the effector caspases. Once activated, effector caspases cleave a small subset of proteins in the cell and it is the cumulative effect of these cleavage events that accounts for most of the physical characteristics of apoptosis.

The signalling pathways that mediate apoptosis are tightly regulated by positive and negative signals that determine if a cell will survive or die. The anti-apoptotic members of the Bcl-2 family play a major role in regulation of apoptosis induced by a variety of different stimuli and members of the inhibitor of apoptosis protein (IAP) family and the heat-shock protein (Hsp) family provide negative apoptotic signalling by interfering with key components of the apoptotic machinery. Although caspases are considered as main executioners in apoptosis, also other proteases have been suggested to play an important role in cell death. Several lines of evidence suggest that many non-caspase proteases like cathepsins, calpains, and serine proteases can act in concert with caspases in apoptosis signalling pathways.

Type 1 Plasminogen Activator Inhibitor

Plaminogen Activator Inhibitor-1, PAI-1, belongs to the serpin (serine protease inhibitor) superfamily, which includes inhibitors of a variety of serine proteases including the two other PAI proteins, PAI-2 and PAI-3. The PAI-1 gene codes for a ˜50 kDa glycosylated protein, which is secreted from the cell. PAI-1 is the primary inhibitor of the plasminogen activation system, a proteolytic cascade involved in various physiological and pathological processes including wound healing, inflammation, vascular thrombolysis, tumour invasion and angiogenesis. PAI-1 inhibits the two types of plasminogen activators, the tissue-type Plasminogen Activator (t-PA) and urokinase-type Plasminogen Activator (u-PA). Both activators are capable of catalysing the conversion of the inactive zymogen plasminogen to the active protease plasmin, which can degrade most extracellular proteins, a mechanism involved in cancer dissemination.

Being an inhibitor of the plasminogen activation system one would expect that high levels of PAI-1 would inhibit tumour progression. However, high levels of PAI-1 in tumours are correlated with poor prognosis in a number of tumours, including carcinoma of the breast, ovaries, stomach and kidney. One explanation to this apparent discrepancy is that PAI-1 has a pro-angiogenic effect. However, it has been suggested that the prognostic impact of PAI-1 is not only based on its involvement in angiogenesis. Another explanation is that high levels of PAI-1 contribute to tumour growth by inhibiting apoptosis of tumour cells. In support of this assumption it has been shown that addition of recombinant PAI-1 to tumour cells in culture inhibits apoptosis (Kwaan et al., Br J Cancer. 2000 May; 82(10):1702-8). Furthermore, the apoptosis inhibiting function of PAI-1 could be blocked by co-incubation with a PAI-1 neutralising antibody.

PAI-1 has also been proposed as a predictive marker of resistance to antiestrogen treatment; breast cancer patients with metastatic breast cancer and high tumour tissue PAI-1 content appeared to be more resistant to Tamoxifen treatment as compared with patients with low tumour tissue PAI-1 content (Foekens et al. 1995, J Natl Cancer Inst 87(10):751-756). In contradiction to this observation is the report by Harbeck (Harbeck et al. 2002, Cancer Res 62, 4617-4622), who analysed the relation between tumour tissue PAI-1 content and effect of adjuvant chemotherapeutic treatment of primary breast cancer. They concluded that patients with high tumour tissue PAI-1 content were more likely to benefit from adjuvant chemotherapy.

The various functions of PAI-1 have been studied intensively over the years, however, little is known about how PAI-1 regulates apoptosis.

Tissue Inhibitor of Metalloprotease-1 (TIMP-1)

TIMP-1 is one out a family of four endogenous inhibitors of matrix metalloproteases (MMPs). TIMP-1 is a 25 kDa protein which binds most MMPs with a 1:1 stochiometry. TIMP-1 is pre-sent in various tissues and body fluids and is stored in o-granules of platelets and released upon activation. While the main function of TIMP-1 is supposed to be MMP inhibition, some alternative functions of TIMP-1 have been described, e.g. inhibition of apoptosis and regulation of cell growth and angiogenesis. In addition, some studies have suggested that TIMP-1 may also play a role in the early processes leading to the malignant phenotype.

We have described that measurement of plasma TIMP-1 gives high specificity and high sensitivity in the detection of early stage colorectal cancer (Holten-Andersen et al). In addition, we have shown that measurement of plasma TIMP-1 levels in preoperative or postoperative samples yields strong and stage independent prognostic information in patients with colorectal cancer (Holten-Andersen et al 2000; Holten-Andersen et al., 2005). By measuring TIMP-1 protein in primary breast cancer tissue we and others have shown that high tumour tissue total TIMP-1 levels are associated with shorter patient survival (Schrohl et al., 2003, 2004; Duffy et al).

A role for TIMP-1 in the regulation of apoptosis has been reported and two possible ways for this to happen have been suggested. Both of these support the idea that TIMP-1 inhibits apoptosis.

First, proteolytic degradation of the extracellular matrix leads to loss of differentiation and to apoptosis in mammary epithelial cells both in vitro and in vivo. This indicates that the integrity of the extracellular matrix and the protection of cell-matrix interactions are crucial factors in assuring survival of mammary epithelium. Through the inhibition of MMPs, TIMP-1 is capable of inhibiting degradation of extracellular matrix, thereby possibly inhibiting apoptosis. By crossing mice that over-expressed MMP-3 in the mammary gland with TIMP-1 transgenic mice, Alexander and co-workers demonstrated such apoptosis-inhibitory effect of TIMP-1 observing that apoptosis of the mammary epithelium induced by MMP-3 was reduced by TIMP-1. The mere disintegration of the basement membrane could be responsible for apoptosis induced by proteolytic activity but it has also been speculated that integrin-mediated signalling plays a part.

Second, an apoptosis-inhibitory effect of TIMP-1 that occurs independently of MMP-inhibition has also been demonstrated. In human breast epithelial cells, an ability of endogenous TIMP-1 to inhibit apoptosis induced by abolition of cell adhesion has been demonstrated. This indicates that TIMP-1 is capable of rescuing cells from apoptosis without stabilising extracellular matrix and cell-matrix interactions. The independence of MMP-inhibition in inhibiting apoptosis is supported by the fact that reduced and alkylated TIMP-1, which has lost all MMP-inhibitory effect, still effectively inhibits apoptosis in Burkitt's lymphoma cell lines. The mechanism for this apoptosis-inhibitory effect is not known at present, but different suggestions have been made regarding signalling pathways possibly regulated by TIMP-1. Over-expression of TIMP-1 in human breast epithelial cells is associated with more efficient activation and constitutive activity of focal adhesion kinase (FAK)—a kinase that is normally involved in signalling cell survival. Also, up-regulation of TIMP-1 protein expression in Burkitt's lymphoma cells increased the expression of the anti-apoptotic protein Bcl-XL. It was speculated that the modulation of cell signalling is mediated via interaction of TIMP-1 with a cell surface receptor as the anti-apoptotic effect of TIMP-1 in Burkitt's lymphoma cells was abolished by the neutralisation of secreted TIMP-1 by monoclonal antibodies. This view is further supported by a study that demonstrates binding of TIMP-1 to the surface of malignant breast epithelial cells.

So, TIMP-1 appears to be capable of inhibiting apoptosis via two different mechanisms. Through inhibition of MMPs, TIMP-1 stabilises extracellular matrix and cell-matrix interactions thereby inhibiting apoptosis induced by disintegration of the extracellular matrix. However, TIMP-1 also inhibits apoptosis via a mechanism that is not dependent of its ability to inhibit proteolytic degradation of the extracellular matrix. This latter mechanism may be mediated by the interaction of TIMP-1 with a receptor on the cell surface regulating intracellular signalling pathways involved in apoptosis.

### SUMMARY OF THE INVENTION

It is well known that many types of currently used anti-cancer drugs as well as radiotherapy, induces objective tumour responses by inducing programmed cell death, i.e. apoptosis. In recent years, it has become evident that high tumour tissue levels or high plasma/serum levels of naturally occurring protease inhibitors are associated with short survival of patients with cancer, such as those patients suffering from malignant brain tumour, malignant melanoma, sarcoma, head and neck cancer, gastrointestinal cancer such as gastric, pancreatic, colon, and rectum cancer, carcinoides, lung cancer, breast cancer, gynecological cancer, such as ovary, cervix uteri, and corpus uteri cancer, and urological cancers, such as prostate, renal, and bladder cancer.

The fact that high PAI-1 levels and/or high TIMP-1 levels in the tumour tissue or blood are correlated to poor prognosis in breast cancer, colorectal cancer and also in lung adenocarcinoma suggests that these molecules promote tumour growth, invasion and/or metastasis or confer resistance to anti-neoplastic treatment. As discussed above there are several mechanisms by which PAI-1 and TIMP-1 may play such a promoting role such as protecting the tumour tissue against degradation by the uPA system, participating in cancer cell migration, participating in tumour angiogenesis, interfering with activation/inactivation of growth factors or inhibiting apoptosis. It is likely that some inhibitors of other extracellular proteolytic enzymes and other non-proteolytic matrix-degrading enzymes play a similar role in promoting tumour growth, invasion and/or metastasis.

It is the finding by the present inventors that high PAI-1 levels contribute to tumour growth by a negative regulation of apoptosis. Accordingly, tumours with a high content of PAI-1 will be less sensitive to apoptosis inducing chemotherapeutic drugs. On the other hand, inhibition of PAI-1 will sensitise cancer cells to subsequent apoptosis-inducing chemotherapeutic drugs, while not affecting normal cells, which do not over express PAI-1.

To test the hypothesis that PAI-1 has an apoptosis regulatory function we have established fibrosarcoma cell lines (primary lung fibroblast cultures, in which the fibroblasts undergo spontaneous malignant transformation after 3-4 in vitro passages) from PAI-1 gene-deficient and wild-type mice. Using the clonogenic assay we have tested the cell lines for sensitivity towards various chemotherapeutic drugs (etoposide, vincristine, doxorubicin, cisplatin and ARA-C) all of which are known to induce apoptosis. The PAI-1−/− fibrosarcoma cells were significantly more sensitive than wild type fibrosarcoma cells to treatment with these drugs. We have also tested if the difference in clonogenic potential was due to an increased cytotoxicity of the drugs in the PAI-1−/− fibrosarcoma cells. Indeed, PAI-1−/− fibrosarcoma cells were significantly more sensitive than PAI-1+/+ fibrosarcoma cells to treatment with etoposide. Interestingly, similar results were obtained when apoptosis was induced via the death receptor-signalling pathway by TNFα treatment. Furthermore, these results were repeated in a newly established pair of PAI-1−/− and PAI-1+/+ fibrosarcoma cell lines. In a recent experiment, we stably transfected PAI-1−/− fibrosarcoma cells with a construct mediating PAI-1 expression and showed that these cells indeed expressed PAI-1 protein. By exposing the cells to Etoposide, we could show that they had increased resistance to the drug as compared with plasmid alone transfected cells. All together, these results suggest that PAI-1 protects against apoptosis. However, we have also demonstrated that PAI-1 gene-deficient and wild type mice display equal sensitivity to systemic etoposide treatment measured by weight, and number of white blood cells, thus suggesting a differential sensitivity between cancer cells and normal cells to apoptosis inhibition by PAI-1. This differential sensitivity makes PAI-1 an attractive target in combination with chemotherapeutic drugs i.e. pre-treatment with a PAI-1 inhibitor will increase the cancer cell cytotoxicity with no additional toxicity in normal tissue.

In analogy, we have also established three sets of fibrosarcoma cell lines from TIMP-1 +/+ and TIMP-1 −/− mice and shown that fibrosarcoma cell lines devoid of TIMP-1 are significantly more sensitive to apoptosis inducing cytotoxic treatment, e.g Etoposide. By performing a DNA-histone complex assay, we could demonstrate that Etoposide induced apoptosis.

We have recently gained clinical support for our hypothesis, since we have shown that breast cancer patients with metastatic disease (n=174) and high tumour content of PAI-1 and/or TIMP-1 are resistant to chemotherapeutic drugs (CMF or CEF).

By performing immune histochemistry on paraffin blocks from breast cancer tissue we have shown, by using an anti-TIMP-1 monoclonal antibody, that in approximately 20% of the cases the TIMP-1 immunoreactivity was confined to the tumour cells while in the rest of the cases the TIMP-1 immunoreactivity was localized to the stromal cells in the tumour tissue. Similar results have been published on PAI-1 (Bianchi et al.) Thus, it seems feasible that those high tumour tissue TIMP-1 or PAI-1 cases that show resistance to chemotherapy are those with TIMP-1 and/or PAI-1 immunoreactivity in the cancer cells. The implication of this finding is that performing TIMP-1 and/or PAI-1 immunehistochemistry on archive paraffin blocks could be used to predict resistance to chemotherapy.

Thus, the invention relates to a method of inhibiting the anti-apoptotic function of PAI-1, TIMP-1 and/or other protease inhibitors, thus making the cancer cells more sensitive to apoptosis-inducing anti-cancer treatment such as chemotherapy, endocrine therapy or irradiation in a patient who has been established to have high tumour tissue levels, high blood levels, high urine levels or high saliva levels of PAI-1 and/or TIMP-1, and/or immuneractivity for TIMP-1 and/or PAI-1 in the cancer cells, the method comprising suppressing the anti-apoptotic function of an inhibitor of a protease or of a non-proteolytic matrix-degrading enzyme in malignant tumour tissue or potential malignant tumour tissue without increasing the sensitivity of normal cells to the anti-neoplastic treatment. The latter requires that a differentiated effect of PAI-1 and TIMP-1 inhibition exists between normal and malignant cells, so systemic inhibition of PAI-1 and/or TIMP-1 would only sensitize the malignant cells to subsequent apoptosis-inducing treatment.

We have shown that PAI-1 or TIMP-1 gene-deficiency renders the fibrosarcoma cells sensitive to apoptosis induced by chemotherapeutic drugs and TNFα. Furthermore, we found that PAI-1 gene-deficient and wild-type mice display equal sensitivity to systemic etoposide treatment, thus suggesting a differential sensitivity between cancer cells and normal cells to apoptosis inhibition by PAI-1.

The presently presented methods thus rely on the surprising discovery that it is possible to preferentially inhibit the apoptosis preventive effect of protease inhibitors in malignant tumour tissue and potential malignant tumour tissue without affecting the normal tissue/cells. According to the present invention it is contemplated (although without being limited to any theory) that the high level of protease inhibitors found in some tumours/patient blood samples, is involved in protection of the tumour cells from apoptosis stimuli. Thus, patients exposed to anti-cancer drugs, which act by inducing apoptosis, will not experience any benefit from the treatment if their tumours contain high levels of protease inhibitors.

By the method of the invention the inhibitory effect of the said inhibitor in the malignant tumour tissue or potential malignant tumour tissue is suppressed, inhibited or neutralized, thereby sensitizing the malignant tumour cells but not normal cells to apoptosis-inducing agents.

The invention thus in one aspect relates to a method for improving the effect of an anti-cancer therapy in a patient, the method comprising increasing the susceptibility of malignant cells in the patient to said anti-cancer therapy without substantially increasing the susceptibility of non-malignant cells to said anti-cancer therapy.

This is for instance obtained effecting the suppression of protease inhibitors such as plasminogen activator inhibitor type 1 or tissue type of metalloprotease inhibitors type 1, resulting in the abolishment of the apoptosis inhibitory function of the protease inhibitor and thereby increased tumour cell death by apoptosis inducing anti-cancer treatment. The invention contemplates that systemic inhibition of protease inhibitors does not affect the sensitivity of non-malignant cells to subsequent or concomitant administration of anti-cancer therapy.

Thus, briefly expressed, the present invention relates to a method for enhancing the efficacy of a cancer therapy, wherein the enhancement is effected by interfering with protease inhibitors.

The invention also relates to methods of selecting and identifying compounds that can inhibit the apoptosis preventive effect of protease inhibitors, as well as the use of such compounds in the treatment of cancer patients.

Further, the invention also provides a method for identifying anti-cancer treatment, the effect of which is inhibited by the presence of protease inhibitors, and, in line with this, the invention also provides for a method for identifying anti-cancer treatment, the effect of which is not inhibited by the presence of protease inhibitors.

Finally, the invention includes methods to identify patients who will not respond to conventional cancer therapy, but will be candidates for a protease inhibitor inhibitory treatment in conjunction with conventional anti-cancer treatment.

### LEGENDS TO THE FIGURES

FIG. 1: Graph showing tumourigenicity of wild-type cells and cells from PAI-1 gene deficient animals in wild-type mice and in PAI-1 gene-deficient mice. Growth of tumours formed from PAI-1 −/− transformed fibroblasts (fibrosarcomas) was significantly delayed as these tumours reached a size of 40 mm³ day 74 (median) as compared to PAI-1 +/+ fibrosarcomas which reached this size at day 19 (median), irrespective of the host PAI-1 genotype (p=0.0001).

FIG. 2: Cytotoxic effect of Etoposide on PAI-1−/− and PAI-1+/+ fibrosarcoma cells. PAI-1−/− and PAI-1+/+ fibrosarcoma cells were treated with Etoposide for 48 hours and cytotoxicity was measured as released lactate dehydrogenase activity (% of total activity). Values represent means of three independent experiments ±SD.

FIG. 3: Cytotoxic effect of TNF-α on PAI-1−/− and PAI-1+/+ fibrosarcoma cells. PAI-1−/− and PAI-1+/+ fibrosarcoma cells were treated with TNF-a for 24 hours and cytotoxicity was measured as released lactate dehydrogenase activity (% of total activity). Values represent means of three independent experiments ±SD.

FIG. 4: The effects of various types of cytotoxic drugs on colony formation of wild-type cells and PAI-1 gene-deficient cells, respectively.

FIG. 5: Cytotoxic effect of Etoposide on PAI-1−/− and PAI-1 transfected PAI-1−/− fibrosarcoma cells. PAI-1−/− and transfected PAI-1−/− fibrosarcoma cells were treated with Etoposide for 48 hours and cytotoxicity was measured as released lactate dehydrogenase activity (% of total activity). Values represent means of three independent experiments ±SD.

FIG. 6: Cytotoxic effect of Etoposide on TIMP-1−/− and TIMP-1+/+ fibrosarcoma cells. TIMP-1−/− and TIMP-1+/+ fibrosarcoma cells were treated with Etoposide for 48 hours and cytotoxicity was measured as released lactate dehydrogenase activity (% of total activity). Values represent means of three independent experiments ±SD.

FIG. 7: Immunoreactivity of TIMP-1 in formalin fixed paraffin embedded breast cancer tissue. A: TIMP-1 immunoreactivity is seen in the tumour stromal cells. B: TIMP-1 immunoreactivity is seen in the tumour cells.

### DETAILED DISCLOSURE OF THE INVENTION

In the following a number of terms will be defined in order to characterize the metes and bounds of the present invention.

By the term “suppression” is meant that the apoptosis inhibitory activity of an inhibitor of a protease or of a non-proteolytic matrix-degrading enzyme is significantly reduced i.e. by a degree of at least 25% but preferably reduced by a higher degree such as about 50%, 60%, 70% or even more such as 75%, 80%, 90%, 95%, or 100%. The degree of inhibition of the inhibitor in question by various compounds can be established by use of suitable inhibitory tests.

In the present context, the term “compound” should be understood as in its broadest context as a substance composed of two or more elements, such that the atoms of the elements are firmly linked together and are present in definite proportions, the term thus including conventional chemical compounds as well as e.g. antibodies. Evidently it will be within the skill of the man skilled in the art based upon the teaching in the specification to develop and use tests for the purpose of screening compounds being capable of suppressing the apoptosis inhibitory activity of an inhibitor of a protease or of a non-proteolytic matrix-degrading enzyme.

A “protease inhibitor” or “proteinase inhibitor” (the terms are used interchangeably) is a molecule that inhibits the proteolytic activity of one or several proteases. This means that a protease inhibitor may be specific or that it may exert a more general protease inhibiting effect. For the purposes of the present invention, a protease inhibitor can also denote a molecule that inhibits the activity of a non-proteolytic matrix-degrading enzyme.

A “blocker” of a protease inhibitor is a molecule that suppresses or inhibits the anti-apoptotic effect of a protease inhibitor.

“Apoptosis” is the process defined in the section termed “Background of invention”.

A “preferential increase” in apoptosis of malignant cells means that malignant cells are rendered more susceptible to apoptotic cell death than non-malignant cells having the effect that a known anti-cancer therapeutic regimen, which normally would induce apoptotic cell death in X % of malignant cells and in Y % of relevant normal cells, would now induce apoptotic cell death in (X+n) % of malignant cells and in Y % of relevant normal cells, or the effect that a milder therapeutic regimen will exist that induces apoptotic cell death in X % of malignant cells and in (Y−m) % of relevant normal cells, where both n and m are positive numbers. Another way to put this is to state that the therapeutic index of the anti-cancer therapeutic regimen has been increased.

“Anti-cancer therapy” is a term used for any non-surgical therapeutic regimen that aims at curing or alleviating cancer. Examples are set forth below but anti-cancer therapy can be both chemotherapeutic and/or radiotherapeutic.

“Cytostatic therapy” is chemotherapeutic anti-cancer therapy that involves interference with cell division, i.e. it is a therapeutic regimen where a drug is administered that somehow interacts with the process of mitosis and thereby kills cells that are actively dividing. Cytostatic therapy may be targeted by having the cytostatic substance coupled to a moiety (e.g. an antibody or antibody fragment) that more or less selectively binds to a component in malignant cells

“Cytotoxic therapy” is chemotherapeutic anti-cancer therapy that involves administration of a cytotoxic substance, i.e. a substance that kills cells via a variety of mechanisms. Cytotoxic therapy may be targeted by having the cytotoxic substance coupled to a moiety (e.g. an antibody or antibody fragment) that more or less selectively binds to a component in malignant cells.

“Endocrine therapy” is chemotherapeutic anti-cancer therapy that involves administration of a hormone or a synthetic or naturally occurring mimic of a hormone, or, alternatively, administration of an inhibitor of a hormone or of a hormone receptor or analogue thereof. Also endocrine therapy may be targeted.

“Radiotherapy” is treatment of cancer by subjecting the malignant cells to ionising radiation. Apart from traditional exogenously applied radiation, also radiation therapy may be targeted by having a radionuclide coupled to a targeting moiety.

“Immunotherapy” is cancer-therapy that relies on immune mechanisms. One possibility is administration of antibodies (monoclonal, polyclonal or fragments thereof) that bind to tumour specific antigens in the tumour. Such antibodies may also be able to trigger secondary immunological mechanisms (NK cell activity, complement activation etc). Also administration of activated dendritic cells that can induce cytotoxic T-cell mediated killing of tumour cells is a possibility. Third, it is possible to immunize actively against cancer antigens, thereby inducing active immunity (both cellular and humeral) that targets malignant cells.

### Description of the Preferred Embodiments of the Invention

It is preferred that the therapeutic method of the invention comprises effecting inhibition of the anti-apoptotic effect of protease inhibitor activity of at least one protease inhibitor in the patient, thereby increasing the susceptibility of malignant cells to said anti-cancer therapy relative to the susceptibility of non-malignant cells to said anti-cancer therapy. That is, the method of the invention contemplates a preferential increase in malignant cells' susceptibility to anti-cancer treatment, that is, without changing the sensitivity of the malignant cells without significantly changing the sensitivity of non-malignant cells towards the anti-cancer treatment.

Typically, the inhibition is achieved by administering a blocker of the in vivo anti-apoptotic action of a protease inhibitor to the patient. Protease inhibitors it is of interest to suppress/block are serine protease inhibitors, inhibitors of a metalloprotease, inhibitors of a cysteine protease (thiol protease), inhibitors of an aspartic protease, inhibitors of any other protein degrading enzyme, inhibitors of a heperanase, or inhibitors of any other enzyme participating in degradation of the extracellular matrix, such as non-proteolytic enzyme inhibitors. Preferably, the protease inhibitor is selected from the group consisting of PAI-1, PAI-2, PAI-3, Protease Nexin 1, TIMP-1, TIMP-2, TIMP-3, TIMP-4, Stephin A, Stephin B, and Cystatin C.

The blocker used according to the present invention is suitably selected from the group consisting of a polyclonal antibody, a monoclonal antibody, an antibody fragment, a soluble receptor, a low molecular molecule, a natural product, a peptide, an anti-sense polynucleotide, a ribozyme, and a mimic of an antisense polynucleotide such as an anti-sense LNA or PNA molecule. The art has already shown that monoclonal antibodies are capable of exerting an effect on apoptosis on tumour cells (Kwaan et al.).

In normal practice of the invention, the blocker is administered prior to instigation of the anti-cancer therapy, but depending on the particular kind of anti-cancer therapy and on the pharmacokinetics of the blocker, the blocker can also be administered at the onset or during the anti-cancer therapy.

The blockers may serve as medicaments in their pure form or as pharmaceutical compositions and they may be administered via any of the usual and acceptable methods known in the art, either singly or in combination—as mentioned above, a number of such blockers are already known, and these will be administered in a manner already accepted by regulatory authorities.

The compositions may be formulated to oral administration (including the buccal cavity or sublingually) or by parenteral administration (including intravenous (i.v.), subcutaneous (s.c.), intramuscular (i.m.), intraperitoneal (i.p.)) administration. Other administration routes include epidural, rectal, intranasal or dermal administration or by pulmonary inhalation.

A pharmaceutical composition comprising, as an active principle, a blocker as herein defined, is in admixture with a pharmaceutically acceptable carrier, diluent, vehicle or excipient. Typically, such a pharmaceutical composition will be a dose form selected from the group consisting of an oral dosage form, a buccal dosage form, a sublingual dosage form, an anal dosage form, and a parenteral dosage form such as an intravenous, an intra-arterial, an intraperitoneal, a subdermal, an intradermal or an intracranial dosage form. Especially preferred formulations provide sustained release of blocker.

The compositions may, depending on the particular choice of blocker, be prepared in a manner well known to the field. The compositions are preferably in the form of solid or liquid formulations and methods for their preparation are generally described in “Remington's Pharmaceutical Sciences”, 17th Ed., Alfonso R. Gennaro (Ed.), Mark Publishing Company, Easton, Pa., U.S.A., 1985. Solid formulations are particularly suitable for oral administration, while solutions are most useful for injection or infusion (i.v., s.c., i.m., or i.p.) or intranasal administration.

Such compositions will contain an effective amount of the one or more active blockers together with a suitable carrier in order to provide the dosage in a form compatible with the route of administration selected. The compositions comprises at least one of the blockers together with a physiologically acceptable carrier in the form of a vehicle, a diluent, a buffering agent, a tonicity adjusting agent, a preservative and stabilizers. The excipients constituting the carrier must be compatible with the active pharmaceutical ingredient(s) and preferably capable of stabilizing the blocker without being deleterious to the subject being treated.

Solid compositions may appear in conventional form such as tablets, pills, capsules, suppositories, powders or enterically coated peptides. Liquid compositions may be in the form of solutions, suspensions, dispersions, emulsions, elixirs, as well as sustained release formulations, and the like. Topical compositions may be in the form of plasters or pastes and inhalation compositions may be contained in spray delivery systems.

In a preferred embodiment of the invention depot formulations that include at least one of the blockers are envisioned—this is of special utility in cases where prolonged treatment with the anti-cancer therapy is to take place (e.g. active immunotherapy or other regimens where the anti-cancer effect is not terminated after a few hours). A form of repository or depot formulation may be used so that therapeutically effective amounts of the preparation are delivered into the bloodstream over many hours or days following transdermal injection or deposition. Formulations suitable for sustained release formulations include biodegradable polymers and may consist of appropriate biodegradable polymers, such as L-lactic acid, D-lactic acid, DL-lactic acid, glycolide, glycolic acid, and any isomers thereof. Similarly, the carrier or diluent may include any sustained release material known in the art, such as glyceryl monostearate or glyceryl distearate, alone or mixed with a wax.

Other depot formulations may include, but are not limited to, formulations that include at least one of the blockers disclosed herein combined with liposomes, microspheres, emulsions or micelles and liquid stabilizers.

Aqueous formulations of the blockers may be prepared for parenteral administration by injection or infusion (i.v., s.c., i.m. or i.p.). The blockers can, depending on choice, be utilized as free acids or bases, or as salts. The salts must, of course, be pharmaceutically acceptable, and these will include alkali and metal salts of acidic blockers, e.g., potassium, sodium or magnesium salts. The salts of basic blockers will include salts of halides and inorganic and organic acids, e.g. chloride, phosphate or acetate. Salts of the blockers are readily prepared by procedures well known to those skilled in the art.

The blockers may be provided as liquid or semi-liquid compositions for parenteral administration (e.g. injection, infusion or deposition of slow release depot formulations). The blockers may be suspended or dissolved in an aqueous carrier, for example, in a suitably buffered solution at a pH of about 3.0 to about 8.0, preferably at a pH of about 3.5 to about 7.4, 3.5 to 6.0, or 3.5 to about 5.0. Useful buffers include sodium citrate/citric acid, sodium phosphate/phosphoric acid, sodium acetate/acetic acid, or combinations thereof.

Such aqueous solutions may be rendered isotonic by adjusting the osmotic pressure with a buffering agent, by the inclusion of saline, aqueous dextrose, glycols or by the use of sugars such as lactose, glucose or mannitol and the like.

The compositions may contain other pharmaceutically acceptable excipients such as preservatives, stabilizing agents, and wetting or emulsifying agents as described in “Handbook of Pharmaceutical Excipients”, 3rd Ed., Arthur H. Kibbe (Ed.), Pharmaceutical Press, London, UK (2000). The preservatives may include sodium benzoate, sodium sorbic acid, phenol or cresols and parabens. Stabilizing agents may include carboxymethyl-cellulose, cyclodextrins or detergents.

The preparation may be produced immediately before use from active drug substance and sterile carrier solution. Alternatively, the compositions may be filled into sealed glass vials or ampoules, and if necessary purged with an inert gas, under aseptic conditions and stored until needed. This allows for continued multi-dose therapy but also demands the highest degree of stability of the compound.

Oleaginous formulations of the blockers may be prepared for parenteral administration by injection (s.c., i.m. or i.p.) or topically. The carrier can be selected from the various oils including those of petroleum, animal, vegetable or synthetic origin, e.g., peanut oil, soybean oil, mineral oil, sesame oil, and the like. The compositions may be in the form of solutions or suspensions. Solutions of the blockers may be prepared with the use of detergents and emulsifiers and suspensions may be prepared using powder or crystalline salts. The compositions may be stabilized with preservatives (e.g. butylated hydroxianisole or butylated hydroxytoluene).

For nasal administration by pulmonary inhalation, the formulation may contain one or more blockers, dissolved or suspended in a liquid carrier, in particular, an aqueous carrier, for aerosol application. The carrier may contain auxiliary additives such as solubilizing agents, e.g., propylene glycol, surfactants such as polyoxyethylene, higher alcohol ethers, and absorption enhancers such as lecithin or cyclodextrin and preservatives such as sorbic acid, cresols or parabens.

Topical administration for local application and action of the blockers (convenient if the cancer therapy only provides a local apoptotic effect) may be in the form of pastes prepared by dispersing the active compound in a pharmaceutically acceptable oil such as peanut oil, sesame oil, corn oil or the like. Alternatively, the blockers may be incorporated into patches for dermal administration. Patches may be prepared in a form for iontophoretic application.

Suppositories for transmucosal administration may be in the form of pellets containing an effective amount of a blocker can be prepared by admixing a blocker with a diluent such as carbowax, carnuba wax, and the like, and a lubricant, such as magnesium or calcium stearate.

Solid compositions are preferred for oral administration in the form of tablets, pills, capsules, powders, and the like. Tablets may contain stabilizing buffering agents (e.g. sodium citrate, calcium carbonate and calcium phosphate), disintegrants (e.g. potato or tapioca starch, and complex silicates) binding agents (e.g. polyvinylpyrrolidone, lactose, mannitol, sucrose, gelatin, agar, pectin and acacia) and lubricating agents (e.g. magnesium stearate, stearic acid or sodium lauryl sulfate) as well as other fillers (e.g. cellulose or polyethylene glycols). Liquid formulations for oral administration may be combined with various sweetening agents, flavoring agents, coloring agents, in addition to diluents such as water, ethanol, propylene glycol, glycerin.

The doses the blockers and compositions of the present invention required for the desired therapeutical effects will depend upon on the potency of the blocker, the particular composition used and the route of administration selected. The blocker will typically be administrated in the range of about 0.001 to 10 g per patient per day, preferably from about 1 to about 1000 mg per patient per day, more preferably from about 10 to about 100 mg per patient per day, about 50 mg per patient per day. Dosages for certain routes, for example oral and other non-parenteral administration routes, should be increased to account for any decreased bioavailability, for example, by about 5-100 fold.

The most suitable dosing regimen may best be determined by a medical practitioner for each patient individually. The optimal dosing regimen with the blockers and pharmaceutical compositions depends on factors such as the particular cancer being treated, the desired effect, and the age, weight or body mass index, and general physical conditions of the patient. The administration may be conducted in a single unit dosage form or as a continuous therapy in the form of multiple doses over time. Alternatively, continuous infusion systems or slow release depot formulations may be employed, as the case may be. Two or more blockers or pharmaceutical compositions may be co-administered simultaneously or sequentially in any order. In addition, the blockers may be administered in a similar manner for prophylactic purposes. The best dosing regimen will ultimately be decided by the attending physician for each patient individually.

Preferably, the anti-cancer therapy which is potentiated by means of the present invention comprises subjecting the patient to conditions that induce cell death by apoptosis. Therefore, according to the present invention, the increase in susceptibility of the malignant cells is the consequence of a preferential increase in apoptosis in the malignant cells that are subjected to the anti-cancer therapy.

This renders possible a preferred embodiment, where the anti-cancer therapy is supplemented with treatment of the patient with an anti-cancer drug, the efficacy of which does not depend on expression of protease inhibitors in the tumour tissue—hence, the method of the invention renders possible the choice of an optimized cancer treatment utilising such drugs (that will not suffer the drawback that cancer cells can be transform to an apoptosis insensitive form so as to escape the effect of the drug) while at the same time rendering the existing treatment more rational and effective (because the existing treatment, when relevant, can be combined with the findings of the present invention to preserve the efficacy of apoptosis dependent drugs by blocking protease inhibitors).

Identification of such anti-cancer drugs whose efficacy are not affected by the presence or absence of protease inhibitors in the tumour tissue can be accomplished as discussed infra for identification of similar anti-cancer (and as set forth in Example 9), since the methods for identification of an anti-cancer treatment more generally provides for identification of any means, including drugs, for cancer treatment.

Hence, it is within the scope of the present invention to identify an anti-cancer drug, the efficacy of which is not dependent on presence or absence of apoptosis-inhibiting protease inhibitors, by 1) providing a first population of malignancy-derived cells that are +/+ or +/− for said protease inhibitor, 2) providing a second population of malignancy-derived cells that are −/− for said protease inhibitor, 3) subjecting samples of said first and second populations of cells to anti-cancer treatment with a putative or known anti-cancer drug in the absence and presence of an effective concentration of an agent which blocks the apoptosis protecting effects of the protease inhibitor, 4) determining the degree of apoptosis induced in said samples, and 5) identifying the putative or known anti-cancer drug as one, the efficacy of which is not dependent on presence or absence of apoptosis-inhibiting protease inhibitors if 1) the degree of apoptosis induced in the samples from the first population of cells is not significantly higher in the presence of the agent, and 2) the degree of apoptosis induced in the samples from the second population of cells is not significantly higher in the presence of the agent. More details concerning this choice of method are given below.

The anti-cancer therapy is typically selected from the group consisting of radiation therapy, endocrine therapy, and cytotoxic or cytostatic chemotherapy, immunotherapy, treatment with biological response modifiers, treatment with protein kinase inhibitors, or a combination thereof. That is, any of these different treatment modes can be utilised together with the inventive efficacy-enhancing effect of the use of blockers.

All these possible types of cancer therapy will according to the invention be applicable in their already accepted form as this is practiced by medical practitioners.

In preferred embodiments the cytotoxic or cytostatic chemotherapy is selected from the group consisting of treatment with alkylating agents, topoisomerase inhibitors type 1 and type 2, antimetabolites, tubulin inhibitors, platinoids, and taxanes.

In another preferred embodiment, endocrine therapy is treatment with antiestrogens, aromatase inhibitors, inhibitors of gonadotropins, antiandrogens, antiprogestins, or combinations thereof.

It is advantageous to target malignancies that have a poor prognosis which is correlated to high expression levels of protease inhibitors. It is therefore advantageous to target tumours selected from the group consisting of malignant brain tumour, malignant melanoma, sarcoma, head and neck cancer, gastrointestinal cancer such as gastric, pancreatic, colon, and rectum cancer, carcinoides, lung cancer, breast cancer, gynecological cancer, such as ovary, cervix uteri, and corpus uteri cancer, and urological cancers, such as prostate, renal, and bladder cancer.

Another part of the invention, which is based on the same findings that forms basis for the therapeutic aspects discussed above, is a method for predicting whether a cancer patient will benefit from an anti-cancer therapy, where the efficiency of said anti-cancer therapy depends on tumour tissue expression of protease inhibitors, the method comprising determining whether cells from tumour tissue in the patient expresses any one of a number of preselected protease inhibitors, and establishing that the patient will not benefit from the anti-cancer therapy if any one of said protease inhibitors is expressed beyond a relevant threshold value and establishing that the patient will benefit from the anti-cancer therapy if none of the pre-selected protease inhibitors are expressed beyond their relevant threshold values.

It is also an aspect of the present invention to predict, based on tissue sections or tumour biopsies or the like, whether a cancer patient will benefit from an anti-cancer therapy, where the efficiency of said anti-cancer therapy depends on tumour tissue expression of protease inhibitors. This can be done by determining whether tumour cells in tumour tissue exhibit elevated expression (as judged by determining immunoreactivity) of inhibitors of proteases, i.e. inhibitors such as PAI-1 or TIMP-1. We have, as mentioned above, performed immunohistochemistry on paraffin blocks from breast cancer tissue and have shown, by using an anti-TIMP-1 monoclonal antibody, that in approximately 20% of the cases the TIMP-1 immunoreactivity was confined to the tumour cells while in the rest of the cases the TIMP-1 immunoreactivity was localized to the stromal cells in the tumour tissue. We have concluded that those high tumour tissue TIMP-1 or PAI-1 cases that show resistance to chemotherapy are those with TIMP-1 and/or PAI-1 immunoreactivity in the tumour cells (but not those showing immunoreactivity merely in the stromal cells).

The implication of this finding is that performing TIMP-1 and/or PAI-1 immunohistochemistry (or analysis providing a similar information, cf. below) even on materials such as archive paraffin blocks (or other preserved samples) from the relevant patient could be used to predict resistance to chemotherapy—for instance, in the cases of metastatic or residual disease where it can be shown that originally excised tumour tissue from the patient expressed the protease inhibitors, it will be relevant to either avoid chemotherapy altogether or, preferably, to combine chemotherapy with administration of blockers of the protease inhibitors as taught herein. So, not even does the present invention allow for an evaluation of the patient based on material which can be provided by means of sampling but also for evaluation of the patient based on archive materials such as paraffin sections that date years back. It will be clear to the skilled person, however, that the invention is not in any way limited to use of such archive materials, but also to immunohistochemistry and comparable methods performed on fresh tissue samples, biopsies and the like.

It should be noted that instead of determining immunoreactivity by e.g. immunohistochemistry, it is also possible to determine amplification of genes encoding the inhibitors: Techniques such as fluorescence in situ hybridization (FISH) and chromogenic in situ hybridization (CISH) are exemplary means for detection of such gene amplification. The practical implementations of using, immunohistochemistry, FISH and CISH in analyses on tumour tissue are described in detail in Tanner et al, Am J Pathol. 2000 November; 157(5):1467-72, where HER-2/neu oncogene amplification was assessed by means of all 3 methods.

Antibodies (such as monoclonals) used in immunohistochemistry on paraffin blocks according to the above-indicated embodiment must necessarily be capable of recognizing epitopes that are present in the relevant protease inhibitor when it is in a denatured form. Hence, antibodies binding linear epitopes on the relevant protease inhibitor are preferred.

The preselected list of protease inhibitors includes members that are selected from serine protease inhibitors, inhibitors of a metalloprotease such as TIMP-1 or TIMP-2, inhibitors of a cysteine protease (thiol protease), inhibitors of an aspartic protease, is an inhibitor of any other protein degrading enzyme, inhibitors of a heperanase, and inhibitors of any other enzyme participating in degradation of the extracellular matrix (e.g. a non proteolytic enzyme inhibitor), and preferably the protease inhibitor is selected from the group consisting of PAI-1, PAI-2, PAI-3, Protease Nexin 1, TIMP-1, TIMP-2, TIMP-3, TIMP-4, Stephin A, Stephin B, and Cystatin C.

The prediction method of the invention preferably comprises that the determination of whether cells from tumour tissues in the patient expresses any one of the number of preselected protease inhibitors is performed by measuring on a sample selected from the group consisting of a tumour tissue sample, a blood sample, a plasma sample, a serum sample, a urine sample, a faeces sample, a saliva sample, and a sample of serous liquid from the thoracic or abdominal cavity. The method measuring is conveniently performed by means of DNA level measurement including in situ hybridization, mRNA level measurement such as in situ hybridization, Northern blotting, QRT-PCR, and differential display, and protein level measurement, such as Western blotting, immunohistochemistry, ELISA, and RIA.

In line with the discussion under the therapeutic method of the present invention, the prediction method entails that the anti-cancer therapy (the efficacy of which is predicted) induces cell death by apoptosis. Further as the predictive method may, if deeming the cancer therapy inapplicable or otherwise unwarranted, establish that the patient will benefit from therapy or other drugs that can be found not to depend on the expression level of protease inhibitors if any one of the protease inhibitors are expressed beyond their threshold values.

The predictive method of the invention may conveniently be combined with anti-cancer therapy to provide an improved cancer therapeutic regimen. Thus, the present invention also contemplates a method for anti-cancer treatment of a cancer patient, the method comprising predicting, according to the prediction method of the invention, whether the cancer patient will benefit from an anticancer therapy of choice, where the efficiency of said anti-cancer therapy depends on tumour tissue expression of protease inhibitors, and subsequently

a) subjecting the patient to the anticancer therapy if the prediction provides a positive answer, or

b) subjecting the patient to the improved cancer therapy according to the present invention, if the prediction provides a negative answer.

A positive answer is in this context a statistically based indication that each of the expression levels of the preselected protease inhibitors are below a cut-off value (threshold value) that indicates the minimum expression level of the protease inhibitor in question which will not have a negative influence on the therapeutic efficacy of the anti-cancer treatment.

Consequently, a negative answer is defined by the expression level of at least one of the pre-selected protease inhibitors is beyond such a cut-off value.

One can perform a retrospective/prospective clinical trial, in order to establish the threshold level for a given protease inhibitor so as to determine resistance/sensitivity to anti-cancer treatment of the individual patient:

Retrospectively, stored tumour tissue or blood or urine, or saliva or any other body fluid is obtained from patients who have experienced recurrence of their cancer disease and of whom it is known how they responded to the particular anti-cancer treatment. In the case of tumour tissue, the tissue is homogenized and the level of protease inhibitor is measured in each individual patient sample. Alternatively, immune histochemistry can be performed on fixed paraffin embedded tissue. In the case of body fluids, the sample may be diluted and subsequently, the concentration of protease inhibitor is determined by one of the methods discussed herein.

Concentrations of protease inhibitors in the individual patient is subsequently correlated with the objective response to anti-cancer treatment of this patient. Using logistic regression analysis, and/or Receiver Operating Characteristics (ROC) curves, the sensitivity and specificity obtained by any protease inhibitor concentration can be calculated for the study population.

Similarly, an identical study can be performed where protease inhibitor concentration is determined in prospectively collected samples and then a correlation is made between protease inhibitor concentration and objective response of the individual patient. Using logistic regression analysis or ROC curves the sensitivity and specificity obtained by any proteinase inhibitor concentration can be calculated for the study population

Alternatively, the invention contemplates monitoring a patient undergoing an existing anti-cancer therapy, wherein the monitoring is performed by repeatedly exercising the prediction method of the invention so as to establish, whether the patient will continue to benefit from the existing anticancer therapy, and

a) continuing subjecting the patient to the anticancer therapy if the prediction in the monitoring provides a positive answer, or

b) switching the patient to another anticancer therapy by means of the cancer therapy improvement method of the invention, if the prediction in the monitoring provides a negative answer.

The anticancer therapy used in combination with the method of the invention is advantageously selected from neoadjuvant therapy, adjuvant therapy, and therapy of metastatic disease.

Also encompassed by the present invention are means and methods for identifying agents that are useful in the practice of the present invention.

The present invention relates to a (cell-dependent) method for identifying an agent that blocks the anti-apoptotic effect of a protease inhibitor, the method comprising
providing a first population of malignancy-derived cells that are +/+ or +/− for said protease inhibitor (meaning that the protease inhibitor has a certain expression level) or where the protease inhibitor is provided from an external source,
providing a second population of malignancy-derived cells that are −/− for said protease inhibitor,
subjecting samples of said first and second populations of cells to substantially the same apoptosis-inducing conditions in the absence and presence of a defined concentration of a candidate agent,
determining the degree of apoptosis induced in said samples, and
identifying the candidate agent as an agent that blocks the anti-apoptotic effect of the protease inhibitor if 1) the degree of apoptosis induced in the samples from the first population of cells is significantly higher in the presence of the candidate agent, and 2) the degree of apoptosis induced in the samples from the second population of cells is not significantly higher in the presence of the candidate agent.

Experiments can also be performed in vivo. The experimental animal must be one that does not reject the implanted cells (+/+, +/−, or −/− cells for the proteinase inhibitor), either because the cells are of the same MHC Class as those of the animal, or because the animal is capable of accepting xenogenic grafts (as is the case with nude mice). In addition, mice being +/+, +/− or −/− for the proteinase inhibitor can be used. The person skilled in the art will know what kind of animal model to select when faced with the task of setting up the method of the invention and setting out from a particular cell-type to be implanted. End-points will be cell death as determined by e.g. tumour size following treatment and systemic toxicity by the applied drugs

It is in certain cases also possible to employ a cell-free (cell-independent) system for such an identification: In the event that it is known that a particular effect of a protease inhibitor on a protease and its substrate is relevant for the apoptosis inhibiting effect of the protease inhibitor, a simple assay will constitute addition of the defined concentration of the candidate agent to a system comprising the protease inhibitor, the protease and its substrate combined with measurement of the conversion rate of the substrate. An increase in conversion rate in the presence of the defined concentration indicates that the candidate agent is a putative blocker of protease inhibitor activity.

Advantageously, different defined concentrations of the candidate agent are tested in the possible setups, optionally in parallel, thus allowing for determination of the optimum concentration of the blocker identified by means of the method.

In the cell-dependent method, but also in the cell-independent, it is preferred to supplement with a confirmation step by subsequently reverting −/− cells into +/− or +/+ cells (for the relevant protease inhibitor) and establishing that the reverted cells' susceptibility to apoptosis can be significantly increased by the candidate agent.

In the cell-dependent system, it is preferred that the first population of cells is less susceptible to the apoptosis-inducing conditions than the second population, when both are subjected to the apoptosis inducing conditions in the absence of the candidate agent. It is possible to grow the samples of the first and second population of cells in an experimental animal as well as in culture; the important thing is reproducibility of the conditions of the experimental settings. The model where an experimental animal is used as host for the samples of cells has the advantage that an immediate indication of adverse effects/toxicity is obtained, whereas this would require a separate experimental setup, when using the cell culture system. At any rate, it is preferred to also determine the degree of adverse effects in an experimental animal.

As an alternative to utilising −/− cells as a control, it is possible to utilise a relatively simple animal model when identifying an agent that blocks the anti-apoptotic effect of a protease inhibitor. This method comprises
providing a first population of malignancy-derived cells that are +/+ or +/− for said protease inhibitor or where the protease inhibitor is provided from an external source,
implanting the first population of cells in an experimental animal and allowing them to grow,
subjecting the animal to apoptosis-inducing conditions in the absence and presence of a defined concentration of a candidate agent,
determining the degree of tumour development and/or progression in said animal,
determining the degree of apoptosis-related adverse effects in the animal, and
identifying the candidate agent as an agent that blocks the anti-apoptotic effect of the protease inhibitor if 1) the degree of tumour development is significantly lower in the presence of the candidate agent, and 2) the degree of apoptosis-related adverse effects induced is not significantly higher in the presence of the candidate agent.

This setup is of course similar to the above-described system, where experimental animals are used as hosts for +/+, +/−, and −/− cells, but in this case the effect on the animal's non-malignant cells are used as the indicator of efficiency of the putative blocker.

The present invention also allows for determining whether a particular anti-cancer treatment or anti-cancer drug is in fact dependent on the presence or absence of apoptosis-inhibiting protease inhibitors in the malignancy to be treated. This method entails
providing a first population of malignancy-derived cells that are +/+ or +/− for said protease inhibitor,
providing a second population of malignancy-derived cells that are −/− for said protease inhibitor,
subjecting samples of said first and second populations of cells to substantially the same anti-cancer treatment (namely the anti-cancer treatment to evaluate) or drug in the absence and presence of an effective concentration of an agent which blocks the apoptosis protecting effects of the protease inhibitor (for instance an agent that has been identified by means of the present invention),
determining the degree of apoptosis induced in said samples, and
identifying the anti-cancer treatment or drug as one, the efficacy of which is dependent on presence or absence of apoptosis-inhibiting protease inhibitors if 1) the degree of apoptosis induced in the samples from the first population of cells is significantly higher in the presence of the agent, and 2) the degree of apoptosis induced in the samples from the second population of cells is not significantly higher in the presence of the agent.

In a simpler (but less stringent) version where no negative control is involved, this method entails
providing a first population of malignancy-derived cells that are +/+ or +/− for said protease inhibitor,
subjecting samples of said first population of cells to substantially the same anti-cancer treatment (namely the anti-cancer treatment to evaluate) or drug in the absence and presence of an effective concentration of an agent which blocks the apoptosis protecting effects of the protease inhibitor (for instance an agent that has been identified by means of the pre-sent invention),
determining the degree of apoptosis induced in said samples, and
identifying the anti-cancer treatment or drug as one, the efficacy of which is dependent on presence or absence of apoptosis-inhibiting protease inhibitors if 1) the degree of apoptosis induced in the samples from the first population of cells is significantly higher in the presence of the agent.

Both these experimental setups can of course be used to identify an anti-cancer treatment or drug, the efficacy of which is not dependent on presence or absence of apoptosis-inhibiting protease inhibitors, the method comprising the same initial steps but where one identifies the anti-cancer treatment or drug as one, the efficacy of which is not dependent on presence or absence of apoptosis-inhibiting protease inhibitors if 1) the degree of apoptosis induced in the samples from the first population of cells is not significantly higher in the presence of the agent. Of course, if the test also uses −/− cells, the degree of apoptosis induced in the samples from the second population of cells should not be significantly higher in the presence of the agent.

An example of such a screen for drugs and therapies is set forth in Example 9.

The invention will now be further illustrated by means of the following non-limiting examples; the skilled person will understand how to expand the exemplified embodiments of the invention to a general inventive concept.

### EXAMPLE 1

### Establishment and Characterization of Cell Lines from Gene-Deficient Animals

In order to study the significance of a particular gene product for the sensitivity to anti-neoplastic treatment of cancer, cell lines are, according to the invention, established from wild-type and gene deficient animals. These cell lines can then be used in screening systems, to study the association between efficacy of various types of anti-neoplastic treatment and cell death as well as identification of blockers of the anti-apoptotic function of protease inhibitors.

The present Example describes a method to establish and characterize immortal fibrosarcoma cell lines from PAI-1 gene-deficient mice.

Mice

Mice were kept in isolation on a 12-hour day/night cycle and were fed regular chow. The generation of the PAI-1 −/− mouse has been described previously (Carmeliet P et al., December 1993, J Clin Invest 92(6): 2746-55). The PAI-1 gene-targeted mouse was crossed into the META™/Bom-nu (=META™/Bom nu/nu) (Brunner N et al., 1993, Breast Cancer Res Treat 24, 257-64) athymic nude mouse and were backcrossed for 6-8 generations. The mice used for experiments are pairs of siblings representing homozygous gene-deficient and homozygous wild type mice obtained by heterozygous breeding. In all experiments involving wild-type mice as controls, these were littermates to the PAI-1 deficient mice, and therefore, each separate experiment only included mice from the same backcrossed generation. All experimental evaluations, including measurements of tumour size and blood sampling were performed by an investigator unaware of animal genotype. All experiments were performed according to the guidelines published by the Danish Animal Care Committee.

Primary Cultures

Lungs of 10-13 week old male Meta nu/nu mice are excised and placed in a Petri dish with 10 ml media (M199 with 30% FCS, P/S and 0.15% NaHCO₃). The lungs are mechanically cut into (app. 0.5-1 mm²) small pieces. 3-6 pieces are then placed in a well of a 6 well plate (Nunc, Tissue culture Quality) in one drop of medium (from the cutting) and then placed in a CO₂-incubator at 37° C. for 20 minutes to allow the cells to adhere to the bottom of the well. After 20 minutes 1 ml of medium is added to cover the tissue completely. After another 30 min. another ml of medium is added.

The medium is renewed every 3 days. The wells are inspected at regular intervals and after 3 weeks they are changed to medium without penicillin and streptomycin. After 4-5 weeks wells with outgrow of fibroblasts are harvested and the cells pooled. The cell lines are propagated and expanded. The cells are tested for Mycoplasma contamination and also genotyped to confirm their origin. By the use of RT-PCR and Western blotting, the cells are tested for PAI-1 mRNA expression and protein production, respectively. The cells can now be used at different passages.

Genotyping of Mice and Primary Cultures for PAI-1

In the disrupted allele of the employed PAI-1 deficient mice (Carmeliet et al. 1993, J Clin Invest 92(6): 2746-55 and Carmeliet et al. 1993, 3 Clin Invest 92(6): 2756-60) an XhoI-BamHI neomycin cassette from pPNT (Tybulewicz V L et al., June 1991, Cell 65(7): 1153-63) is inserted in the place of the XhoI-HindIII fragment of the PAI-1 gene. XhoI(911) is located in the promoter region of the PAI-1 gene (Acc.: M33961). The 5′ XhoI-end of the pPNT neomycin cassette consists of a 507 bp EcoRI(417)-TaqI(924) fragment of mouse phosphokinase-1 (PGK) promoter (Acc.: M18735) with its EcoRI site blunt end ligated to HincII of the HincII-XhoI portion of the polylinker of the pIBI30 (Acc.: L08878). MPAI1.1p (TTC ATG CCC TCT GGT CGC TG, SEQ ID NO: 1) upstream and mpai1.2M (CTC CCT CCC TCC CAG TGA CTT G, SEQ ID NO: 2) downstream of the XhoI (911) site amplify a 349 bp stretch specific for the endogenous allele while mPAI1.1p and mPGK2m (GCC TTG GGA AAA GCG CCT C, SEQ ID NO: 3) in the 5′ end of the neomycin cassette PGK promoter amplify a 219 bp stretch specific for the disrupted allele.

Test for Tumourigenicity:

2×10⁶ cells in a volume of 0.2 ml isotonic NaCl (passage 34 PAI-1 −/− and passage 28 PAI-1 +/+) were inoculated subcutaneously into the flank of wild-type or PAI-1 gene-deficient female mice.

PAI-1 −/− cells were inoculated into: PAI-1+/+ mice (n=27) or into PAI-1−/− mice (n=20) and PAI-1 +/+ cells were inoculated into PAI-1+/+ mice (n=10) or into PAI-1 −/− mice (n=10). Mice were observed on a regular basis and tumour incidence and tumour growth were recorded. Resulting tumours were processed for routine histology (HE staining).

Test for Plating Efficiency in Soft Agar

Cells were resuspended in a volume of 3.5 ml medium and to this was added a mixture of agar and medium (Agar: 990 mg Bacto agar and 30 ml of PBS boiled for 60 minutes; medium: M199 and FCS 10% heated to 37° C.; mixture: 10 ml of agar and 90 ml of medium mixed and heated in water bath to 37° C.). Gentle aspiration was repeated using a 1 ml syringe to achieve single cell suspension.

1 ml was then plated in triplicate in Petri dishes upon a feeder layer of SRBC (see below).

When the agar had solidified, 1 ml of medium was added on top. 18-24 dishes were placed on plastic trays and two Petri dishes containing water were placed on each tray for conditioning. Cells were grown in a CO₂-incubator (CO₂: 7.5%) at 37° C. and 100% humidity. Colonies (>64 cells) were counted after 3 weeks.

Cells were aspirated using a 1 ml syringe to achieve single cell suspension, and were mixed with Nigrosin 0.1% (1:1). Vital cells were counted after 8 minutes.

Preparation of Media:

M199 was supplemented with 10% FCS, 25 mM Hepes buffer, 1.9 mM L-glutamin, 10 ml 7.5% NaHCO₃, 50 U/ml Penicillin and 50 μg/ml Streptomycin.

Preparation of SRBC (Sheep Red Blood Cells) Agar Plates

Agar:

1200 mg agar and 100 ml sterile H₂O was boiled for 1 hour, and then placed on water bath 37° C. for 5 minutes.

SRBC:

Sheep Blood was centrifuged at 5500 rpm and supernatant was removed; the red blood cells were then washed twice in isotonic saline.

Complete Feeder Layer:

Agar, SRBC, media and supplements were mixed (to the final concentrations in solution shown): Earle's MEM without L-Glutamine (×0.55), Earle's MEM amino acids (×0.547), Earle's MEM vitamins (×0.55), L-glutamine (1.1 mM), Penicillin (27.4 U/ml), Streptomycin (27.4 U/ml), Glucose (0.03%), Sodium Bicarbonate (0.06%), 2-mercaptoethanol (3 μl/l), Agar (0.5%), SRBC (0.03 ml/ml).

1 ml of this mixture was then placed in a Petri dish and left to solidify for 1 hour at room temperature. Kept at 5° C. and used within 1 week after manufacturing.

Materials:

PBS (pH 7.5) was composed of: NaCl 8.0 g/l; KCl 0.2 g/l; Na₂HPO₄, 2H₂O 1.44 g/l; KH₂PO₄ 0.2 g/l and sterilized in an autoclave.

Bacto agar was obtained from Bie & Berntsen (Roedovre, Denmark), Sheep Blood in Alsevers liquid (1:1) was from The State Serum Institute (Copenhagen, Denmark).

All reagents were obtained from Gibco (Taastrup, Denmark).

Results

Primary Cultures

After 3-4 days cells started to sprout from the primary explants. Following approximately 1-2 passages (3-4 weeks in culture), the cells underwent a crisis (lasting up to 8 weeks) following which they grew as monolayers with a doubling time of approximately 1.5 days. Both types of cells have now been propagated for more than 40 passages.

RT-PCR showed expression of PAI-1 mRNA in wild-type cells only and Western blot confirmed that only wild-type cells had PAI-1 protein.

Growth of Tumours In Vivo

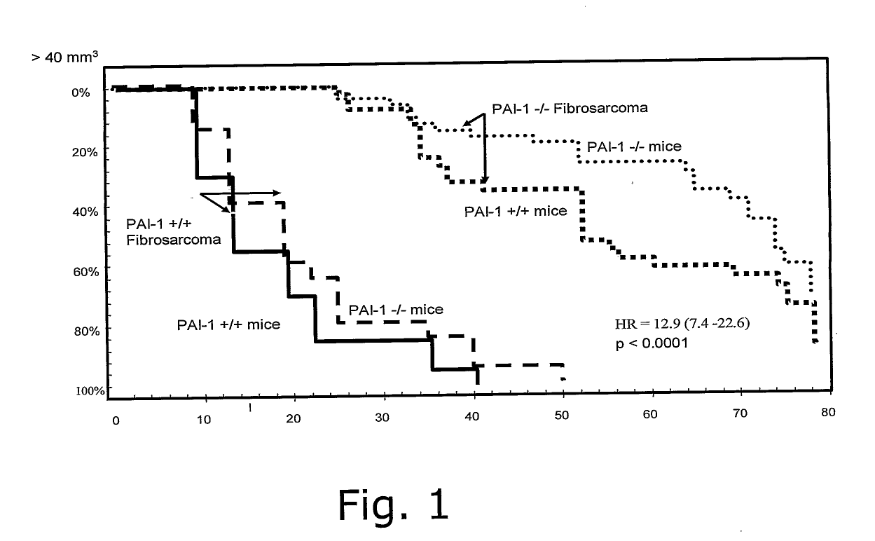

Both wild-type cells and cells from PAI-1 gene deficient animals formed tumours in wild-type mice and in PAI-1 gene-deficient mice. However, cells from PAI-1 gene-deficient mice had a longer lag-period. Cf. FIG. 1.

All tumours had the histological appearance of fibrosarcomas and both cell lines formed colonies in soft agar, but PAI-1 +/+ had a 10 times higher plating efficacy than PAI-1 −/−.

Discussion

This Example shows that mouse fibroblasts undergoes spontaneous malignant transformation when cultured in vitro. The resulting transformed cells form tumours in mice and form colonies in soft agar, both strong indications of their malignant transformation. In addition, the cells lines appeared to be immortal, since they could be propagated for many passages. Thus, wild-type and gene-deficient continuous cell lines are available for experimentation.

By an equivalent experimental procedure, pairs of cell lines from other types of gene-deficient and wild-type mice can be established.

### EXAMPLE 2

### Test of Fibrosarcoma Cell Lines for Chemosensitivity Using Clonogenic Assay

Several of the protease inhibitors have been described to protect cells against apoptosis. Since many types of anti-neoplastic treatment kill cells by inducing apoptosis, it would be anticipated that cell lines being devoid of the expression of protease inhibitors would be more sensitive to apoptosis-inducing anti-neoplastic treatment.

This example describes the testing for chemosensitivity of wild-type and PAI-1 gene-deficient cell lines to a variety of cytotoxic drugs.

Cell Lines

The cell lines described in Example 1 were used. The cells were in passage 35 (wild-type cells) and passage 50 (PAI-1 gene deficient cells).

Clonogenic Assay

Drug (35 μl) and cells (0.35 ml) were mixed, and to this was added 3.15 ml of mixture of agar and medium (Agar: 990 mg Bacto agar and 30 ml of PBS boiled for 60 minutes; medium: M199 and FCS 10% heated to 37° C.; mixture: 10 ml of agar and 90 ml of medium mixed and heated in water bath to 37° C.). Gentle aspiration was repeated using a 1 ml syringe to achieve single cell suspension.

1 ml was then plated in triplicate in Petri dishes upon a feeder layer of SRBC (see separate paragraph for preparation of these).

When the agar had solidified, 1 ml of media was added on top. 18-24 dishes were placed on plastic trays and two Petri dishes containing water were placed on each tray for conditioning. Cells were grown in a CO2-incubator (CO₂ 7.5%) at 37° C. and 100% humidified. Colonies (>64 cells) were counted after 3 weeks.

Preparation of Drugs:

The experimental drugs were dissolved in media×300 the final concentration.

Cells:

Cells were aspirated using a 1 ml syringe to achieve single cell suspension, and were mixed with Nigrosin 0.1% (1:1). Viable cells were counted after 8 minutes.

Preparation of Medium:

M199 was supplemented with FCS 10%, Hepes buffer 25 mM, L-glutamin 1.9 mM, Penicillin 50 U/ml and Streptomycin 50 μg/ml.

Preparation of SRBC (Sheep Red Blood Cells) Agar Plates

Agar:

1200 mg agar and 100 ml sterile H2O was boiled for 1 hour, and then placed on water bath 37° C. for 5 minutes.

SRBC:

Sheep Blood was centrifuged at 5500 rpm and supernatant was removed; the red blood cells were then washed twice in isotonic saline.

Complete Feeder Layer:

Agar, SRBC, media and supplements were mixed (to the final concentrations in solution shown): Earle's MEM without L-Glutamine (×0.55), Earle's MEM amino acids (×0.547), Earle's MEM vitamins (×0.55), L-glutamine (1.1 mM), Penicillin (27.4 U/ml), Streptomycin (27.4 U/ml), Glucose (0.03%), Sodium Bicarbonate (0.06%), 2-mercaptoethanol (3 μl/l), Agar (0.5%), SRBC (0.03 ml/ml).

1 ml of this mixture was then placed in a Petri dish and left to solidify for 1 hour at room temperature. Kept at 5° C. and used within 1 week after manufacturing.

Materials:

PBS (pH 7.5) was composed of: NaCl 8.0 g/l; KCl 0.2 g/l; Na₂HPO₄, 2H₂O 1.44 g/l; KH₂PO₄ 0.2 g/l and sterilized in an autoclave.

Bacto agar was obtained from Bie & Berntsen (Rødovre, Denmark), Sheep Blood in Alsevers liquid (1:1) was from the State Serum Institute (Copenhagen, Denmark).

All reagents were obtained from Gibco (Taastrup, Denmark).

Results

The effects of various types of cytotoxic drugs on colony formation of wild-type cells and the PAI-1 gene-deficient cells, respectively, are shown in FIG. 4. It is seen that PAI-1 deficient cells are more sensitive than wild type cells to all of the drugs applied.

Discussion

This example shows that cells devoid of PAI-1 expression are more sensitive to apoptosis-inducing agents than cells expressing PAI-1.

An equivalent experimental design can be used to test the sensitivity of other pairs of wild-type and gene-deficient cells against anticancer drugs.

### EXAMPLE 3

### Effect of PAI-1 Gene Deficiency on In Vivo Toxicity of Etoposide (VP-16)

When administering cytotoxic drugs systemically to a cancer patient, both the cancer cells and the normal cells in the rest of the body will be exposed to the toxic effects of the drug. If one sensitizes cells to the cytotoxic effect of a drug, such a sensitisation will potentially affect both cancer cells and normal cells.

In this example we show that while sensitivity to cytotoxic drugs is enhanced in cancer cells devoid of PAI-1 expression, this is not the case when studying toxicity in normal cells.

Toxicity Experiments In Vivo

We investigated the sensitivity of the intact mouse by a “comparison” of sensitivity between the PAI-I +/+ and −/− mice in terms of weight loss.

Furthermore blood was sampled on the day of expected nadir (day 3 (WBC)) and day 5, with hematological evaluation. The experimental drug used in this study was etoposide.

Mice: META™/Bom nu/nu; PAI-1+/+ and PAI-1 −/−. Size (gram): 24-30 grams females and males. (See Example 1 for further characterization).

Mice were anaesthetized with 0.15 ml hypnorm/dormicum (2.5 mg/ml; 1.25 mg/ml) before blood sampling because WBC has been found to be elevated when taking blood samples from tail veins in un-anaesthetized mice. 4 male and 6 female PAI-1 +/+ mice and 8 male and 6 female PAI-1 −/− mice received treatment with 75 mg/kg etoposide i.p.

As control, 4 male and 4 female PAI-1 +/+ mice and 5 male and 4 female PAI-1 −/− mice received vehicle i.p.

Drugs/Test Articles:

Etoposide, purchased from Pharmacia A/S, Denmark, Batch number: T309A. The drug was administered i.p., freshly made, relative to body weight, in a NaCl solvent (Batch 3036111) according to the following table:

| | | | | | |
|---|---|---|---|---|---|
| | Number of | | | | |
| Group | mice | Male: | Male: | Female: | Female: |
| number | per group: | PAI-1 +/+ | PAI-1 −/− | PAI-1 +/+ | PAI-1 −/− |

Etoposide dose (mg/kg) or vehicle or saline:

| | | | | | |
|---|---|---|---|---|---|
| 1 | 4 | 75 | | | |
| 2 | 8 | | 75 | | |
| 3 | 6 | | | 75 | |
| 4 | 6 | | | | 75 |
| 5 | 4 | Vehicle | | | |
| | | control* | | | |
| 6 | 5 | | Vehicle | | |
| | | | control* | | |
| 7 | 4 | | | Vehicle | |
| | | | | control* | |
| 8 | 4 | | | | Vehicle |
| | | | | | control* |

*as (˜etoposide 75 mg/kg)

Results

In Vivo.

Drug induced effect on weight loss

| | mg/kg | Gen- | Time (hours) | | | | | |
|---|---|---|---|---|---|---|---|---|
| Genotype | etoposide | der | 0 | 22.0 | 44.5 | 67.0 | 93.5 | 120.0 |
| PAI-1 +/+ | 75 | M | 100 | 97.6 | 94.9 | 92.9 | 91.0 | 91.8 |
| PAI-1 −/− | 75 | M | 100 | 99.1 | 96.3 | 96.4 | 95.6 | 97.0 |
| PAI-1 +/+ | 75 | F | 100 | 101.2 | 98.3 | 98.2 | 98.4 | 97.7 |
| PAI-1 −/− | 75 | F | 100 | 99.9 | 99.1 | 99.6 | 98.7 | 100.3 |
| PAI-1 +/+ | 0 | M | 100 | 99.9 | 99.0 | 102.9 | 101.8 | 102.3 |
| PAI-1 −/− | 0 | M | 100 | 101.9 | 103.0 | 105.8 | 103.8 | 104.2 |
| PAI-1 +/+ | 0 | F | 100 | 101.8 | 103.7 | 105.0 | 103.7 | 103.1 |
| PAI-1 −/− | 0 | F | 100 | 103.0 | 104.5 | 105.8 | 103.6 | 106.0 |

There is a significant loss of weight in etoposide treated mice.

By statistical normalisation to initial weight, the effect of treatment was a significant weight loss for the treated mice compared to the untreated (p<0.0001), while no difference was seen between genotypes (p=0.30) or gender (p=0.41).

Drug Induced Effect on White Blood Cells (WBC)

Etoposide suppresses white blood cell count (WBC) in both genotypes with nadir on day 3 (p=0.0003), but there is no difference (p=0.99) in the response from PAI-1 +/+ and PAI-1 −/− mice, or between males or females (p=0.58).

Discussion

This example demonstrates that while the sensitivity of cancer cells to cytotoxic drugs is enhanced by PAI-1 gene-deficiency, normal cells in the live animal as indicated by drug induced death, weight loss and wbc counts are not sensitised to the cytotoxic effect of cytotoxic drugs by the induced PAI-1 gene-deficiency. Thus, the present findings indicates that concomitant treatment with a cytotoxic drug and with a blocker of a protease inhibitor will increase the therapeutic index of the cytotoxic drug. In a broader sense, this example indicates that a blocker of the anti-apoptotic function of a protease inhibitor can be administered systemically prior to the administration of cytotoxic drugs without resulting in increased systemic toxicity.

### EXAMPLE 4

### Quantisation of Apoptosis Induced by VP-16 or Tumour Necrosis Factor-α (TNF-α) in Wild Type and PAI-1 Gene-Deficient Fibrosarcoma Cells

It was tested if the difference in clonogenic potential was due to an increased cytotoxicity of the drugs in the PAI-1−/− fibrosarcoma cells.

Materials and Methods

The damage of the plasma membrane is classically evaluated as a parameter for cell death. In vivo the plasma membrane of apoptotic cells persists until the cell is phagocytised. In contrast, necrotic cell death results in leakage of cytoplasm to the extracellular environment, which leads to an inflammatory response. Under cell culture conditions, cells that have been subjected to an apoptotic stimulus will initially die by apoptosis, but later shift into secondary necrosis due to lack of phagocytosis in the cell culture. Cytotoxicity or cell lysis can be measured by determining the release of lactate dehydrogenase (LDH) in the culture supernatant. For this purpose the “Cytotoxicity detection kit” (Roche, Mannheim, Germany) was employed.

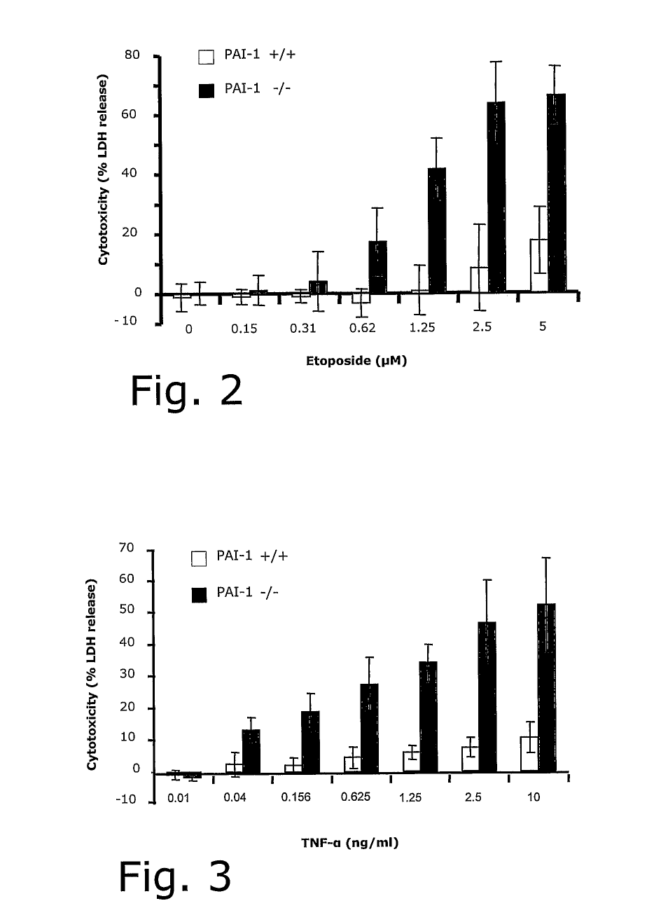

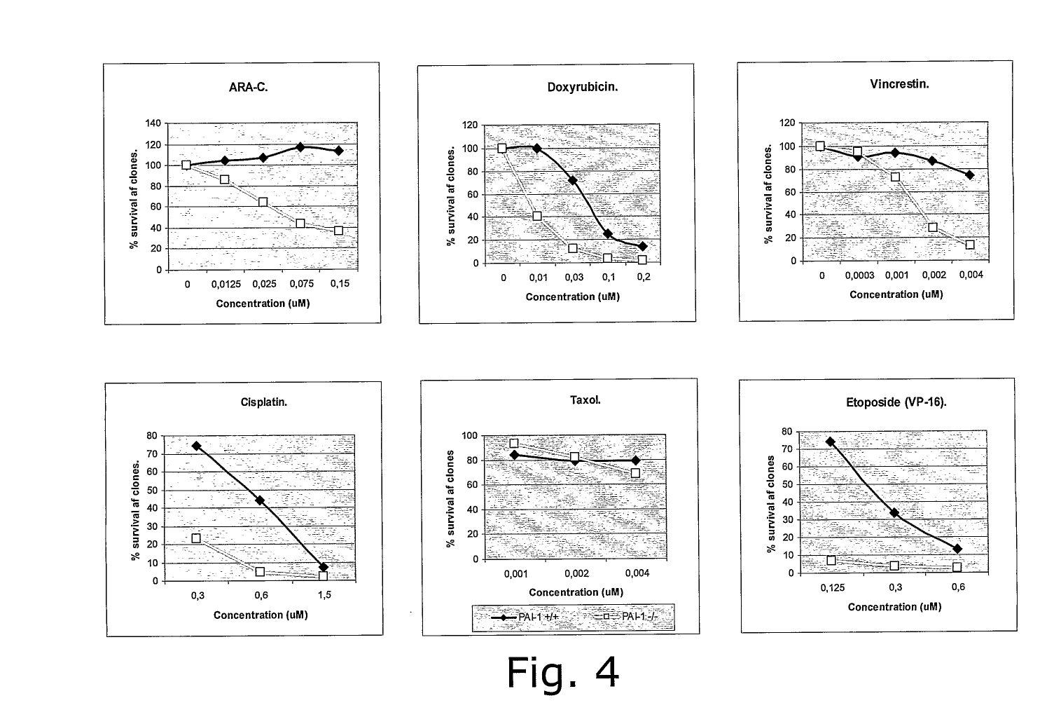

PAI-1−/− and PAI-1+/+ fibrosarcoma cells were seeded in a 96-well microtiter plate (2500 cells/well). After 24 hours, cells are treated with TNF-α and etoposide for 24 h and 48 h, respectively, as indicated in FIGS. 3 and 2. Fifty μl of culture supernatant (total: 200 μl) is transferred to a new 96-well microtiter plate and mixed with 50 μl of a substrate mix. The remaining supernatant is discarded, and the remaining intact cells are lysed by addition of 200 μl of lysis buffer (1% Triton-X100 in CM). After 30 min lysis at 37° C., 50 μl of lysate is transferred to a new 96-well microtiter plate and mixed with 50 μl of a substrate mix. The cell culture supernatant and lysate are incubated for 10 min with the substrate mix protected from light. The absorbance is measured on a spectrophotometer at λ₁=490 nm and reference λ₂=650 nm. The amount of released LDH in % is related to total amount:

$$Cytotoxicity ( \% LDH release ) = \frac{{LDH}_{supernatant}}{Total LDH ( {LDH}_{supernatant} + {LDH}_{lysate} )} \times 100 \%$$

Results

To analyse if PAI-1 gene-deficiency renders the fibrosarcoma cells sensitive to apoptosis, the PAI-1−/− and PAI-1+/+ fibrosarcoma cells were treated with Etoposide and TNF-α. FIGS. 3 and 2 shows that Etoposide and TNF-α induced a dose-dependent cell lysis of both PAI-1−/− and PAI-1+/+ fibrosarcoma cells. However, PAI-1−/− fibrosarcoma cells are significantly more sensitive to etoposide and TNF-α treatment than PAI-1−/− fibrosarcoma cells. As shown in FIG. 2, treatment with 1.25 μM of etoposide induced 1% LDH release from PAI-1+/+ fibrosarcoma cells whereas 41.10% LDH was released from the PAI-1−/− fibrosarcoma cells, and as shown in FIG. 3, treatment with 2.5 ng/ml TNF-α induced 7.3% LDH release from PAI-1+/+ fibrosarcoma cells whereas 46.3% LDH was released from the PAI-1−/− fibrosarcoma cells. These results were reproduced in a newly established pair of PAI-1−/− and PAI-1+/+ fibrosarcoma cell lines.

Conclusion:

All together, this example shows that lack of PAI-1 gene expression makes the malignant cells more sensitive to apoptosis-inducing agents. This finding is in full agreement with the findings of Examples 2 and 3, and underscores that it will be possible to supplement apoptosis-inducing anti-cancer treatment so as to increase the therapeutic index (TD50/ED50) of the relevant anti-cancer treatment.

### EXAMPLE 5

### Quantifying Apoptosis Induced by VP-16 in Wild Type and TIMP-1 Gene-Deficient Fibrosarcoma Cells

It was tested if Etoposide induced an increased cytotoxicity in the TIMP-1−/− fibrosarcoma cells as compared to wild-type cells.

Materials and Methods

The damage of the plasma membrane is classically evaluated as a parameter for cell death. In vivo the plasma membrane of apoptotic cells persists until the cell is phagocytised. In contrast, necrotic cell death results in leakage of cytoplasm to the extracellular environment, which leads to an inflammatory response. Under cell culture conditions, cells that have been subjected to an apoptotic stimulus will initially die by apoptosis, but later shift into secondary necrosis due to lack of phagocytosis in the cell culture. Cytotoxicity or cell lysis can be measured by determining the release of lactate dehydrogenase (LDH) in the culture supernatant. For this purpose the “Cytotoxicity detection kit” (Roche, Mannheim, Germany) was employed.

TIMP-1−/− and TIMP-1+/+ fibrosarcoma cells (see above method for establishment of fibrosarcoma cells from mice) were seeded in a 96-well microtiter plate (2500 cells/well). After 24 hours, cells are treated with etoposide for 24 h. Fifty μl of culture supernatant (total: 200 μl) is transferred to a new 96-well microtiter plate and mixed with 50 μl of a substrate mix. The remaining supernatant is discarded, and the remaining intact cells are lysed by addition of 200 μl of lysis buffer (1% Triton-X100 in CM). After 30 min lysis at 37° C., 50 μl of lysate is transferred to a new 96-well microtiter plate and mixed with 50 μl of a substrate mix. The cell culture supernatant and lysate are incubated for 10 min with the substrate mix protected from light. The absorbance is measured on a spectrophotometer at λ₁=490 nm and reference λ₂=650 nm. The amount of released LDH in % is related to total amount:

$$Cytotoxicity ( \% LDH release ) = \frac{{LDH}_{supernatant}}{Total LDH ( {LDH}_{supernatant} + {LDH}_{lysate} )} \times 100 \%$$

Results

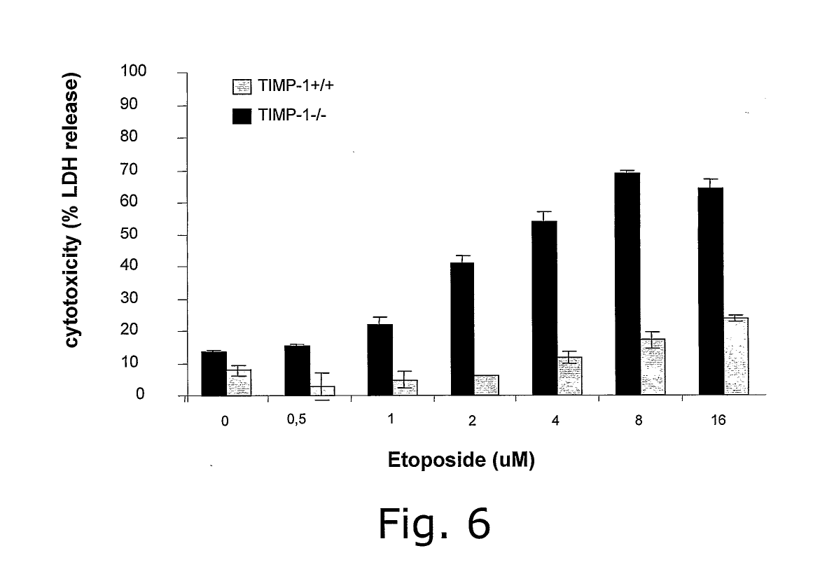

To analyse if TIMP-1 gene-deficiency renders the fibrosarcoma cells sensitive to apoptosis, the TIMP-1−/− and TIMP-1+/+ fibrosarcoma cells were treated with Etoposide. FIG. 6 shows that Etoposide induced a dose-dependent cell lysis of both TIMP-1−/− and TIMP-1+/+ fibrosarcoma cells. However, TIMP-1−/− fibrosarcoma cells are significantly more sensitive to etoposide treatment than TIMP-1−/− fibrosarcoma cells. These results were reproduced in two additional pairs of TIMP-1−/− and TIMP-1+/+ fibrosarcoma cell lines.

Conclusion:

This example shows that lack of TIMP-1 gene expression renders the malignant cells more sensitive to apoptosis-inducing agents and underscores that it will be possible to supplement apoptosis-inducing anti-cancer treatment so as to increase the therapeutic index (TD50/ED50) of the relevant anti-cancer treatment.

### EXAMPLE 6

### High Through-Put Screening for Chemicals and Natural Products, Which can Inhibit the Anti-Apoptotic Function of a Protease Inhibitor

A: A simple Cell Based Assay to Screen for Blockers of PAI-1

PAI-1+/+ fibrosarcoma cells are seeded in a 96-well microtiter plate (2500 cells/well). After 24 hours, cells are treated with chemical compounds or natural products one hour prior to treatment with chemotherapeutic drugs. Cell death are analysed by the cytotoxicty detection kit (Roche, Germany) as described in Example 4. A hit is defined as a chemical compound or natural product which sensitises the PAI-1+/+ fibrosarcoma cells to treatment with chemotherapeutic drugs. The hits identified in the screening are subsequently tested on PAI-1−/− fibrosarcoma cells to verify that the sensitising effect of the hits is due to an inhibition of PAI-1. Thus, compounds and natural products that sensitises PAI-1+/+ fibrosarcoma cells but have no effect on PAI-1−/− cells are selected for further analyses.

A similar experimental setup can be used with other pairs of wild-type and gene-deficient fibrosarcoma cell lines, e.g. TIMP-1 cells

B: An Alternative Cell Based Assay to Screen for Blockers of Anti-Apoptotic Function of PAI-1

Recombinant PAI-1 has been demonstrated to inhibit etoposide and camptothecin induced apoptosis of tumour cells, when rPAI-1 was added directly to the cells in culture (Kwaan et al., 2000, BJC). This protective effect can be used to screen for compounds (natural or synthetic) that inhibit the anti-apoptotic effect of PAI-1. PC-3 cells are seeded in a 96-well microtiter plate. After 24 hours, cells are treated with recombinant human PAI-1 (rhPAI-1) one hour prior to treatment with chemotherapeutic drugs. Controls are: 1) cells treated with chemotherapeutic drugs without addition of rhPAI-1 and compounds or natural products and 2) cells treated with chemotherapeutic drugs in combination with rhPAI-1 but without compounds or natural products. After 48 hours of treatment, cell death is analysed by the Cytotoxicty detection kit (Roche, Germany) as described in Example 4. Compounds or natural products that sensitises the cells to apoptosis induced by chemotherapeutic drugs are selected for further analysis. A similar experimental setup can be used with other pairs of wild-type and gene-deficient fibrosarcoma cell lines, e.g. TIMP-1 cells and recombinant protease inhibitor, e.g. TIMP-1.

C: A Cell-Free Assay to Screen for Putative Blockers of Anti-Apoptotic Function of PAI-1

Recombinant PAI-1 is coated on the bottom of a multiwell plate. Alternatively, an antibody is used to link the rPAI-1 to the plastic surface of the multiwell. Test material with a potential blocker is added and the mixture is incubated. The wells are then washed and subsequently, labelled uPA or tPA is added and the mixture is incubated. The wells are now being washed and a detection system for the labelled molecules are applied. If the test material contained a blocker of PAI-1/uPA or PAI-1/tPA, no label will be detected.

A similar experimental setup can be used for other proteinase inhibitors, e.g. TIMP-1 and Matrix metalloproteinases.

### EXAMPLE 7

### Confirmation by Reversion of Genotype

To confirm that the increased sensitivity to apoptosis of PAI-1 gene-deficient fibrosarcoma cells is indeed due to the lack of PAI-1 expression it was tested whether the sensitive phenotype can be reverted by reintroduction of PAI-1 expression.

Materials and Methods

Transfection of PAI-1−/− fibrosarcoma cells was performed by the use of Lipofectamine 2000 (Roche) according to the manufactures instructions employing 2×10⁵ cells. After 2 days the cells were seed in tissue culture flasks and 100 μg/ml Hygromycin was added to the medium to select for transfected cells in pooled population. Experiments were performed 2 months after transfection. The sensitivity to apoptosis was measured by treating the cells with etoposide and TNF-α and detecting the LDH release as described in Example 4.

Results

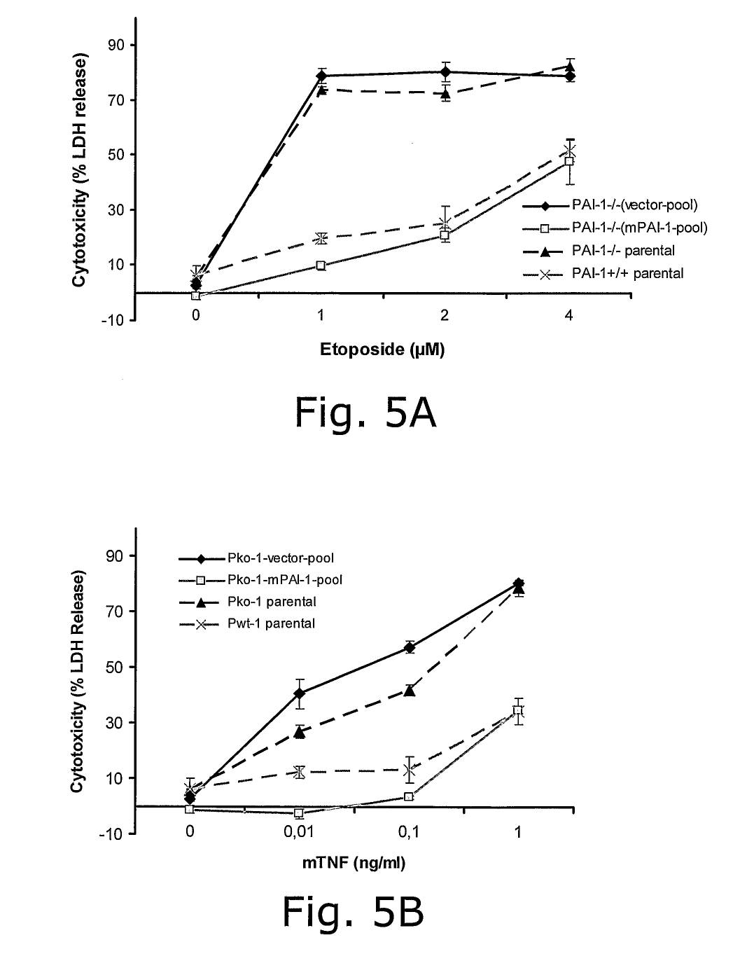

To analyse if ectopic expression of murine PAI-1 in PAI-1−/− fibrosarcoma cells renders the fibrosarcoma cells less sensitive to apoptosis, PAI-1−/− fibrosarcoma cells were stably transfected with an expression plasmid containing murine PAI-1 cDNA. After selection of transfected cells, the transfected and parental fibrosarcoma cells were treated with Etoposide and TNF-α. FIGS. 5a and 5b shows that Etoposide and TNFα induces cell lysis of both transfected and parental fibrosarcoma cells. However, PAI-1−/− cells ectopically expressing mPAI-1 (PAI-1−/−(PAI-1 pool)) are significantly more resistant to etoposide and TNF-a treatment than PAI-1−/− (vector pool) and parental PAI-1−/− fibrosarcoma cells. The PAI-1−/−(PAI-1 pool) cells exhibited almost the same resistance to etoposide and TNFα induced cell death as did the parental PAI-1+/+ fibrosarcoma cells. In concordance with this result, PAI-1−/−(PAI-1 pool) and PAI-1+/+ fibrosarcoma cells have an similar expression levels of PAI-1 (data not shown).

Conclusion:

This example shows that reintroduction of PAI-1 expression renders malignant cells more resistant to apoptosis-inducing agents. This finding confirms that the sensitive phenotype of PAI-1−/− fibrosarcoma cells is due to lack of PAI-1 gene-expression. This supports the hypothesis of an anti apoptotic function of PAI-1.

### EXAMPLE 8

### The Predictive Value of TIMP-1 and PAI-1 in Patients with Metastatic Breast Cancer

Introduction

A large number of breast cancer patients will experience recurrence of disease. When diagnosed with metastatic disease, these patients are offered anti-cancer treatment, which can be cytotoxic therapy, endocrine treatment, radiotherapy, or other treatment modalities. The objective response rate to treatment in patients with metastatic breast cancer is usually as low as 50-60% and very few patients are cured. Thus, since only 50-60% of the patients respond to treatment, a large proportion is treated with no effect. However, this group of treated non-responders still suffer from side effects associated with the treatment.

Thus, a method for identifying patients that will not benefit from a specific treatment will be of great importance for the quality of life of the patient and will also have socio-economic value. Ideally, testing for efficacy of different treatments should be possible; in that case a patient could be offered the treatment type most efficacious at an early stage.

Retrospectively, we have studied the predictive value of TIMP-1 and PAI-1 concentrations in tumour tissue from primary breast tumours. 174 patients were included in the study, all had metastatic breast cancer and all had received chemotherapy with cytotoxic drugs.

Materials and Methods

Patients

Tissue samples were collected as part of a larger study, which was approved by the medical ethical committee of the Erasmus University Rotterdam, The Netherlands (protocol no. MEC 02.953). Inclusion criteria for this large study were as follows: Patients were diagnosed with primary breast cancer between 1978 and 1992, had no metastatic disease at the time of diagnosis, had no previous diagnosis of carcinoma (except for basal cell skin carcinoma and cervical cancer stage I), and had no evidence of disease within 1 month of primary surgery. Patients with inoperable T4 tumours (staging according to the International Union Against Cancer TNM tumour-node-metastasis classification) and patients who received neoadjuvant treatment before surgery were excluded. Tissue specimens that were obtained form a biopsy were not included. Furthermore, patients admitted to the institute more than 100 days after primary surgery and patients with distant metastasis at the time of primary surgery (M1 patients) were excluded. Selection of samples was based on the availability of stored cytosol extracts (in liquid nitrogen), which remained after routine estrogen receptor (ER) and progesterone receptor (PgR) analyses.

The 174 samples included in the present study were selected from the total group of samples based on the presence of metastatic disease, which had been treated with cytotoxic therapy.

Median age of the patients at the time of surgery was 47 years (range 24-79). 116 (67%) were lymph node-positive (including 2 patients with unknown lymph node status) and 58 (33%) were lymph node-negative. 93 patients (53%) were premenopausal and 81 (47%) were postmenopausal at time of start of 1^{st} line chemotherapy. T₁ tumours (<2 cm) were pre-sent in 42 patients (24%), T₂ tumours (2-5 cm) in 101 patients (58%), T₃ tumours (>5 cm) in 16 patients (9%), and operable T₄ tumours in 11 patients (6%). 4 patients had a tumour of unknown T status. Pathological examination was performed as follows: Tumour size was recorded as the largest diameter of the tumour. The differentiation grade was based on histological and cellular characteristics, as stated in the reports of the regional pathologist, and it is not based on a central pathological review of all tumour samples, and thus it reflects daily practice. The local pathologists classified the tumours as well, moderately, or poorly differentiated. Lymph nodes were examined histologically to confirm the number of nodes with tumour involvement. The histological differentiation grade was poor in 112 patients (64%), moderate in 10 patients (6%), and unknown for 52 patients (30%). Adjuvant chemotherapy (mainly cyclophosphamide/methotrexate/5-fluorouracil, CMF) was given to 37 patients (mainly premenopausal patients), whereas 25 patients received adjuvant hormonal therapy (mainly postmenopausal patients), either alone (24 patients) or in combination with chemotherapy (1 patient).

25 patients had loco-regional disease relapse, 18 had a supra-clavicular relapse, 116 suffered from distant metastases, 7 had spread of disease to the contra lateral breast, and 8 patients had metastases to the regional lymph nodes. The dominant site of relapse was soft tissue in 30 patients, bone in 31 patients, and viscera in 113 patients. Median age at the start of chemotherapy for metastatic disease was 50 years. 94 patients (54%) received CMF for metastatic disease and 80 patients (46%) received an anthracycline-containing regimen. The median time to progression from the start of 1^{st} line chemotherapy was 5 months and the median survival time was 14 months. Overall, the objective response rate to chemotherapy was 37%.

Tumour Tissue Extraction

Tumour tissue samples were stored in liquid nitrogen and pulverized in the frozen state with a microdismembrator as recommended by the European Organization for Research and Treatment of Cancer (EORTC) for processing of breast tumour tissue for cytosolic ER and PgR determinations. The resulting tissue powder was suspended in EORTC receptor buffer (10 mM dipotassium chloride EDTA, 3 mM sodium azide, 10 mM monothioglycerol, and 10% v/v glycerol, pH 7.4). The suspension was centrifuged for 30 min at 100,000×g to obtain the supernatant fraction (cytosol).

TIMP-1 ELISA

Total levels of TIMP-1 were determined by a sandwich-format ELISA: Immunoassay plates (Nunc Maxisorp, Nunc, Denmark) were coated with 100 μL of sheep polyclonal anti-TIMP-1 antibody, diluted to 4 mg/L in 0.1 M carbonate buffer, pH 9.5) overnight at 4° C. The wells were then rinsed twice with 200 μL of Pierce Superblock (Pierce Chemicals) diluted 1:1 in PBS. Washing of the wells was then performed five times with PBS containing 1 g/L Tween-20. After washing, wells were incubated for 1 h at 30° C. with duplicates of the tissue extracts. Extracts, previously diluted to 1 mg protein/ml in EORTC receptor buffer, were further diluted 22-fold in sample dilution buffer (50 mM phosphate, pH 7.4, 10 mg/ml bovine serum albumin (Fraction V, Sigma-Aldrich, Steinheim, Germany) and 0.1% v/v Tween-20). On each assay plate a series of standards (diluted in assay dilution buffer as described above) consisting of seven dilutions (5, 3, 2, 1, 0.5, 0.25, and 0.1 ng/ml, respectively) of human recombinant TIMP-1 was included in duplicate together with a duplicate of blank wells (assay dilution buffer only). After binding of TIMP-1 the wells were washed five times with PBS including 1 g/L Tween-20 and TIMP-1 was detected using a specific anti-TIMP-1 monoclonal antibody, which detects both free TIMP-1 and complexes of TIMP-1 and various MMPs [MAC 15]. The monoclonal antibody was diluted in sample dilution buffer to a concentration of 0.5 mg/L and incubation was at 30° C. for 1 h with 100 μL of diluted antibody per well. Plates were then washed five times with PBS containing 1 g/L Tween-20 and incubated for 1 h at 30° C. with 100 μL per well of a rabbit-anti-mouse polyclonal antibody conjugated with alkaline phosphatase (DAKO, Glostrup, Denmark). This antibody was diluted 1:2000 in sample dilution buffer. After incubation, plates were washed five times with PBS and 1 g/L Tween-20 and three times with pure water. 100 μL of freshly made p-nitro phenyl phosphate (Sigma) substrate solution (1.7 g in 0.1 M Tris-HCl, pH 9.5, 0.1 M NaCl, 5 mM MgCl) was added to each well and plates were read at 405 nm in an absorbance plate reader. Readings were performed automatically every 10 minutes for 1 h.

The assay has been thoroughly validated for measurement of TIMP-1 concentrations in cytosolic extracts of tumours (Schrohl et al. 2003, Mol Cell Proteomics. 2(3): 164-72).

PAI-1 ELISA

Concentrations of PAI-1 were determined by means of a sandwich-format. Microtiter plates (Greiner, Alphen a/d Rijn, the Netherlands) were coated with a solution of polyclonal anti-PAI-1 antibody (#395-G, American Diagnostica, Greenwich, Conn., 100 μL per well, 2 μg/L). After removal of the coating solution, wells were incubated with 100 μL of tissue extracts or PAI-1 standards. Extracts had previously been diluted to a concentration of 1 mg protein/ml and were further diluted 20-fold. PAI-1 standards covered the range 0.05-5.0 ng/ml and were obtained from American Diagnostica (#1090). Incubation was overnight at 4° C. in a humidified chamber. After this, plates were washed four times and incubated for 1 h at 23° C. with a culture supernatant of an anti-PAI-1 monoclonal antibody (HD-PAI-1 14.1 ref.) diluted 1:20. This antibody detects active as well as inactive forms of PAI-1 and also PAI-1 in complex with urokinase, tissue-type plasminogen activators, and vitronectin. After incubation, plates were washed and treated with 100 μL of peroxidase-conjugated goat-anti mouse anti-body per well for 1 h at 23° C. The amount of PAI-1 was detected using the 1,2 phenylenediamine reaction (DAKO, Glostrup, Denmark) and after 10 minutes the reaction was stopped with H₂SO₄ and absorbance was measured at 490 nm.

Controls were included at all plates (pooled human breast tumour cytosols) and were used for calculation of inter-assay variations; intra-assay variation was determined as well (Foekens et al.).

Determination of Total Protein Concentration

Total cytosolic protein was quantified by means of the Coomassie brilliant blue method (BioRad Laboratories, CA) with human serum albumin as a standard. Protein concentrations were used for normalization of concentrations of TIMP-1 and PAI-1 (ng TIMP-1 or PAI-1 per mg of total protein).

Results

Concentrations of TIMP-1 and PAI-1 were normalized against total protein concentration of the tissue extract (ng TIMP-1 or PAI-1 per mg of total protein).

To allow for categorization of tumours into TIMP-1-low and TIMP-1-high ones and into PAI-1-low and PAI-1-high ones, cut points were identified by means of isotonic regression analysis.

Using these cut points, 18 patients were classified as having TIMP-1-high tumours (156 had TIMP-1-low tumours) and 25 patients had PAI-1-high tumours (149 had PAI-1-low tumours). We then looked at the response to chemotherapy in the following groups:
a) Tumour tissue TIMP-1 and PAI-1 low (142 patients)
b) Tumour tissue TIMP-1 low and PAI-1 high or TIMP-1 high and PAI-1 low or both high (32 patients)

Response to chemotherapy was evaluated as response (complete or partial) or no response (progressive or stable disease). Results were as follows (p<0.001):

| Group | a (low/low) | b (high/low or high/high) |
|---|---|---|
| Response | 44% (62/142) | 6% (2/32) |
| No response | 56% (80/142) | 94% (30/32) |

Thus, in the group of patients having high tumour tissue levels of either TIMP-1, PAI-1 or of both the response rate to chemotherapy (CMF or anthracyclines) was only 6%. In the group of patients that had both low TIMP-1 and PAI-1 levels in their tumour, the response rate was 44%.

Conclusions

This study indicates that patients with breast cancer whose primary tumour expresses high levels of TIMP-1 and/or PAI-1 has a minimal chance of responding to chemotherapy with CMF or anthracycline-based regimens in the metastatic settings. However, patients with low levels of TIMP-1 and PAI-1 in the primary tumour constitute a subgroup in which response to chemotherapy with CMF or anthracyclines can be anticipated.

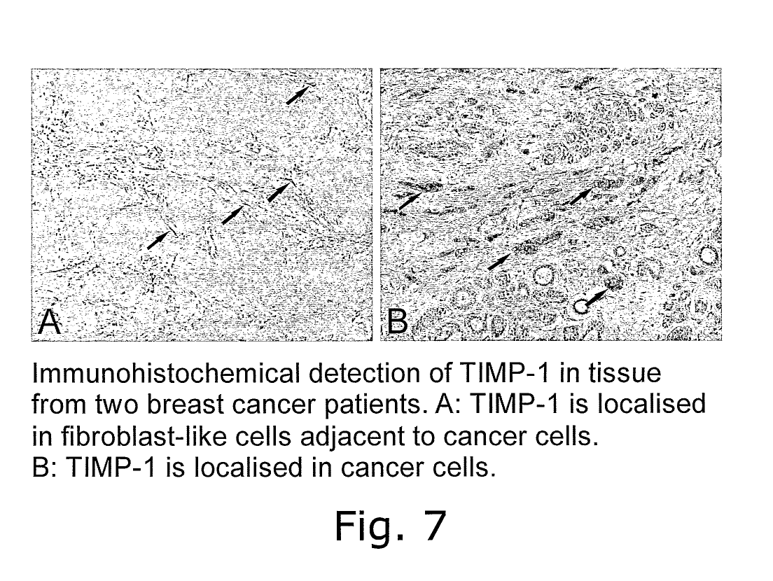

As mentioned in the above general description of the invention, it would also have been possible to determine whether cancer cells from the same patients express TIMP-1 or PAI-1 by means of immunohistochemistry or FISH or CISH—FIG. 7 demonstrates the striking difference in the localisation pattern of TIMP-1 in tumour tissue, where TIMP-1 is localised in stromal cells (FIG. 7A) and cancer cells (FIG. 7B), thus demonstrating the easy readout from an immunohistochemical analysis of tumour tissue: Those patients exhibiting immunoreactivity towards PAI-1 or TIMP-1 (or, alternatively, amplification of the corresponding genes as shown by FISH or CISH) would in such cases be regarded as non-responders to a chemotherapeutic regimen with CMF or anthracycline.

### EXAMPLE 9

### Testing Drugs for Dependence on Protease Inhibitors

PAI-1 +/+ or PAI-1 −/− fibrosarcoma cells are seeded in a multi-well dish. Anticancer drug is added and 24 to 48 hours later, the effect as determined by LDH release (see example 4) is measured. If PAI-1 −/− cells are more sensitive to the treatment as compared to PAI-1 +/+ cells, the efficacy of the drug in question can be concluded to be dependent on PAI-1, while if a similar sensitivity is seen in the two cell lines, the effect of the anticancer drug is independent of PAI-1. A similar experimental setup can be used to test other pairs of proteinase-inhibitor wild-type and gene deficient fibrosarcoma cell lines.

1. A method for improving the effect of an anti-cancer therapy in a patient, the method comprising increasing the susceptibility of malignant cells in the patient to said anti-cancer therapy without substantially increasing the susceptibility of non-malignant cells to said anti-cancer therapy.

2. The method according to claim 1, comprising effecting inhibition of the anti-apoptotic effect of a protease inhibitor activity of at least one protease inhibitor in the patient, thereby increasing the susceptibility of malignant cells to said anti-cancer therapy relative to the susceptibility of non-malignant cells to said anti-cancer therapy.

3. The method according to claim 2, wherein inhibition is achieved by administering a blocker of the in vivo anti-apoptotic action of a protease inhibitor to the patient.

4. The method according to claim 3, wherein the protease inhibitor is a serine protease inhibitor, is an inhibitor of a metalloprotease, is an inhibitor of a cysteine protease (thiol protease), is an inhibitor of an aspartic protease, is an inhibitor of any other protein degrading enzyme, is an inhibitor of a heperanase or is an inhibitor of any other enzyme participating in degradation of the extracellular matrix.

5. The method according to claim 3, wherein the blocker is selected from the group consisting of a polyclonal antibody, a monoclonal antibody, an antibody fragment, a soluble receptor, a low molecular molecule, a natural products, a peptide, an anti-sense polynucleotide, a ribozyme, and a mimic of an antisense polynucleotide.

6. The method according to claim 3, wherein the blocker is administered prior to instigation of the anti-cancer therapy.

7. The method according to claim 3, wherein the blocker is administered at the onset or during the anti-cancer therapy.

8. The method according to claim 3, wherein the blocker is administered as part of a pharmaceutical composition that includes a pharmaceutically acceptable carrier, vehicle or diluent.

9. The method according to claim 8, wherein the pharmaceutical composition is in a dosage form selected from the group consisting of an oral dosage form; a buccal dosage form; a sublingual dosage form; an anal dosage form; and a parenteral dosage form.

10. The method according to claim 8, wherein administration is via a route selected from the group consisting of the parenteral route; the peritoneal route; the oral route; the buccal route; the sublinqual route; the epidural route; the spinal route; the anal route; and the intracranial route.

11. The method according to claim 1, wherein the anti-cancer therapy comprises subjecting the patient to conditions that induce cell death by apoptosis.

12. The method according to claim 1, wherein the increase in susceptibility of the malignant cells is the consequence of a preferential increase in apoptosis in the malignant cells that are subjected to the anti-cancer therapy.

13. The method according to claim 1, wherein the anti-cancer therapy is supplemented with treatment of the patient with an anti-cancer drug, the efficacy of which does not depend on expression of protease inhibitors in the tumour tissue.

14. The method according to claim 1, wherein the anti-cancer therapy is selected from the group consisting of radiation therapy, endocrine therapy, and cytotoxic or cytostatic chemotherapy, immunotherapy, treatment with biological response modifiers, treatment with protein kinase inhibitors, or a combination thereof.

15. The method according to claim 14, wherein the cytotoxic or cytostatic chemotherapy is selected from the group consisting of treatment with alkylating agents, topoisomerase inhibitors type 1 and type 2, antimetabolites, tubulin inhibitors, platinoids, and taxanes.

16. The method according to claim 14, wherein the endocrine therapy is treatment with antiestrogens, aromatase inhibitors, inhibitors of gonadotropins, antiandrogens, antiprogestins, or combinations thereof.

17. The method according to claim 1, wherein the anti-cancer therapy targets a malignant neoplasm selected from the group consisting of malignant brain tumour, malignant melanoma, sarcoma, head and neck cancer, gastrointestinal cancer, carcinoides, lung cancer, breast cancer, gynecological cancer, and urological cancers.

18. The method according to claim 17, wherein elevated protease inhibitor expression is correlated with poor prognosis.

19. A method for predicting whether a cancer patient will benefit from an anti-cancer therapy, where the efficiency of said anti-cancer therapy depends on tumour tissue expression of protease inhibitors, the method comprising determining whether cells from tumour tissue in the patient expresses any one of a number of preselected protease inhibitors, and establishing that the patient will not benefit from the anti-cancer therapy if any one of said protease inhibitors is expressed beyond a relevant threshold value and establishing that the patient will benefit from the anti-cancer therapy if none of the preselected protease inhibitors are expressed beyond their relevant threshold values.

20. The method according to claim 19, wherein the preselected protease inhibitors are selected from the group consisting of the protease inhibitors defined in claim 4.

21. The method according to claim 19, wherein determination of whether cells from tumour tissues in the patient expresses any one of the number of preselected protease inhibitors is performed by measuring on a sample selected from the group consisting of a tumour tissue sample, a blood sample, a plasma sample, a serum sample, a urine sample, a faeces sample, a saliva sample, and a sample of serous liquid from the thoracic or abdominal cavity.

22. The method according to claim 21, wherein measuring is performed by means of DNA level measurement, mRNA level measurement or protein level measurement.

23. The method according to claim 19, wherein the anti-cancer therapy induces cell death by apoptosis.

24. The method according to claim 22, wherein measuring is performed by means of DNA level measurement or protein level measurement and performed on archive material from the patient.

25. The method according to claim 24, wherein the DNA level measurement is selected from fluorescent in situ hybridization and chromogenic in situ hybridization.

26. The method according to claim 25, wherein the protein level measurement is immunohistochemistry.

27. A method for anti-cancer treatment of a cancer patient, the method comprising predicting, according to the method of claim 19, whether the cancer patient will benefit from an anticancer therapy, where the efficiency of said anti-cancer therapy depends on tumour tissue expression of protease inhibitors, and subsequently
a) subjecting the patient to the anticancer therapy if the prediction provides a positive answer, or
b) subjecting the patient to the improved cancer therapy according to claim 1, if the prediction provides a negative answer.

28. A method for anti-cancer treatment of a cancer patient, the method comprising monitoring a patient undergoing an existing anti-cancer therapy, wherein the monitoring is performed by repeatedly exercising the prediction according to the method of claim 19, whether the patient will continue to benefit from the existing anticancer therapy, and
a) continuing subjecting the patient to the anticancer therapy if the prediction in the monitoring provides a positive answer, or
b) switching the patient to another anticancer therapy by means of the method according to claim 1, if the prediction in the monitoring provides a negative answer.

29. The method according to claim 27, wherein the anti-cancer therapy is selected from neoadjuvant therapy, adjuvant therapy, and therapy of metastatic disease.

30. A method for identifying an agent that blocks the anti-apoptotic effect of a protease inhibitor, the method comprising
providing a first population of malignancy-derived cells that are +/+ or +/− for said protease inhibitor or where the protease inhibitor is provided from an external source,
providing a second population of malignancy-derived cells that are −/− for said protease inhibitor,
subjecting samples of said first and second populations of cells to substantially the same apoptosis-inducing conditions in the absence and presence of a defined concentration of a candidate agent,
determining the degree of apoptosis induced in said samples, and
identifying the candidate agent as an agent that blocks the anti-apoptotic effect of the protease inhibitor if 1) the degree of apoptosis induced in the samples from the first population of cells is significantly higher in the presence of the candidate agent, and 2) the degree of apoptosis induced in the samples from the second population of cells is not significantly higher in the presence of the candidate agent.

31. The method according to claim 30, wherein different defined concentrations of the candidate agent are tested, optionally in parallel.

32. The method according to claim 30, wherein the result is subsequently confirmed by reverting −/− cells into +/− or +/+ cells and establishing that the reverted cells' susceptibility to apoptosis can be significantly increased by the candidate agent.

33. The method according to claim 30, wherein the first population of cells is less susceptible to the apoptosis-inducing conditions than the second population in the absences of the candidate agent.

34. The method according to claim 27, wherein the samples of the first and second population of cells are grown in an experimental animal.

35. The method according to claim 27, wherein the samples of the first and second population of cells are grown in culture.

36. The method according to claim 34, wherein the degree of adverse effects in the animal is also determined.

37. A method for identifying an agent that blocks the anti-apoptotic effect of a protease inhibitor, the method comprising
providing a first population of malignancy-derived cells that are +/+ or +/− for said protease inhibitor or where the protease inhibitor is provided from an external source,
implanting the first population of cells in an experimental animal and allowing them to grow,
subjecting the animal to apoptosis-inducing conditions in the absence and presence of a defined concentration of a candidate agent,
determining the degree of tumour development and/or progression in said animal,
determining the degree of apoptosis-related adverse effects in the animal, and
identifying the candidate agent as an agent that blocks the anti-apoptotic effect of the protease inhibitor if 1) the degree of tumour development is significantly lower in the presence of the candidate agent, and 2) the degree of apoptosis-related adverse effects induced is not significantly higher in the presence of the candidate agent.

38. A method for identifying an anti-cancer treatment the efficacy of which is dependent on presence or absence of apoptosis-inhibiting protease inhibitors, the method comprising
providing a first population of malignancy-derived cells that are +/+ or +/− for said protease inhibitor,
providing a second population of malignancy-derived cells that are −/− for said protease inhibitor,
subjecting samples of said first and second populations of cells to substantially the same anti-cancer treatment in the absence and presence of an effective concentration of an agent which blocks the apoptosis protecting effects of the protease inhibitor,
determining the degree of apoptosis induced in said samples, and
identifying the anti-cancer treatment as one, the efficacy of which is dependent on presence or absence of apoptosis-inhibiting protease inhibitors if 1) the degree of apoptosis induced in the samples from the first population of cells is significantly higher in the presence of the agent, and 2) the degree of apoptosis induced in the samples from the second population of cells is not significantly higher in the presence of the agent.

39. A method for identifying an anti-cancer treatment the efficacy of which is not dependent on presence or absence of apoptosis-inhibiting protease inhibitors, the method comprising
providing a first population of malignancy-derived cells that are +/+ or +/− for said protease inhibitor,
providing a second population of malignancy-derived cells that are −/− for said protease inhibitor,
subjecting samples of said first and second populations of cells to substantially the same anti-cancer treatment in the absence and presence of an effective concentration of an agent which blocks the apoptosis protecting effects of the protease inhibitor,
determining the degree of apoptosis induced in said samples, and
identifying the anti-cancer treatment as one, the efficacy of which is not dependent on presence or absence of apoptosis-inhibiting protease inhibitors if 1) the degree of apoptosis induced in the samples from the first population of cells is not significantly higher in the presence of the agent, and 2) the degree of apoptosis induced in the samples from the second population of cells is not significantly higher in the presence of the agent.

40. Use of a blocker of a protease inhibitor for the preparation of a pharmaceutical preparation for enhancing the effect of anti-cancer therapy.

41. The use according to claim 40, wherein the protease inhibitor is selected from the group consisting of the protease inhibitors defined in claim 4.

42. The use according to claim 40, wherein the blocker is selected from the group consisting of the blockers defined in claim 5.

43. The use according to claim 40, wherein the anti-cancer therapy comprises subjecting the patient to conditions that induce cancer cell death by apoptosis.

44. The use according to claim 40, wherein the blocker induces a preferential increase in apoptosis in the malignant cells compared to non-malignant cells when the patient is subjected to the anti-cancer therapy.

45. The use according to claim 40, wherein the anti-cancer therapy is selected from the group consisting of radiation therapy, endocrine therapy, and cytotoxic or cytostatic chemotherapy, or a combination thereof.

46. The use according to claim 45, wherein the cytotoxic or cytostatic chemotherapy is selected from the group consisting of treatment with the agents defined in claim 1.

47. The use according to claim 45, wherein the endocrine therapy is selected from the group consisting of treatment with the agents defined in claim 15.

48. The method according to claim 3, wherein the protease inhibitor is selected from the group consisting of PAI-1, PAI-2, PAI-3, Protease Nexin 1, TIMP-1, TIMP-2, TIMP-3, TIMP-4, Stephin A, Stephin B, and Cystatin C.

49. The method according to claim 22, wherein the MRN level measuring is performed by in situ hybridization, Northern blotting, QRT-PCR, or differential display.

50. The method according to claim 22, wherein the protein level measurement is performed by Western blotting, Immunohistochemistry, ELISA or RIA.

51. The method according to claim 5, wherein the mimic of an antisense polynucleotide is an anti-sense LNA or PNA molecule.

52. The method according to claim 9, wherein the parenteral dosage form is an intravenous, an intra-arterial, an intraperitoneal, a subdermal, an intradermal, an intramuscular, or an intracranial dosage form.

53. The method according to claim 10, wherein the parenteral route is the intradermal, the subdermal, the intra-arterial, the intravenous, or the intramuscular route.

54. The method according to claim 17, wherein the gastrointestinal cancer targeted by the anti-cancer therapy is gastric, pancreatic, colon or rectum cancer.

55. The method according to claim 17, wherein the gynecological cancer targeted by the anti-cancer therapy is ovary, cervix uteri, or corpus uteri cancer.

56. The method according to claim 17, wherein the urological cancer targeted by the anti-cancer therapy is prostate, renal or bladder cancer.

57. The method according to claim 24 wherein the archive material from the patient is paraffin block comprising tumour tissue.
